SEQUENCE LISTING (23 sequences)

SEQ ID NO: 1 - DNA, 876 bp, Artificial Sequence
  Description of Artificial Sequence Synthetic polynucleotide
atgaagccgcatattagtattctgggtgcaggtcgtatgggcagtgccctggtgaaagcatttctgcagaatgaatataccaccaccgtttggaatcgtacccgtgcacgttgtgaaccgctggcagcagcaggcgcccgtattgccgatagcgttcgcgatgcagtgcagaccgccagcgtggttattgtgaatgtgaatgattatgataccagcgatgccctgctgcgccaggatgaagtgacccaggaactgcgcggtaaagttctggtgcagctgaccagcggcagtccgaaactggcccgcgaacaggccacctgggccagacgtcatggtattgattatctggatggtgcaattatggccaccccggatctgattggtcgtccggattgtaccctgctgtatgccggcccgaaagcactgtatgataaacatcaggccgttctggcagcactgggtggcaatacccagcatgttagtgaagatgaaggtcatgcaagcgcactggatagtgccattctgtttcagctgtggggtagcctgtttagtggtctgcaggccgccgcaatttgtcgtgcagaaggcattgccctggatgcactgggtccgcatctggaagcagtggccgccatgattcagtttagcatgaaagatctgctgcagcgtattcagaaagaacagtttggtgcagataccgaaagcccggcaaccctggatacccataatgttgcctttcagcatctgctgcatctgtgcgaagaacgtaatattcatcgcgccctgccggaagcaatggatgcactgattcagaccgcacgcaaagccggtcatggccaggatgattttagtgttctggcacgttttctgcgttaa

SEQ ID NO: 2 - protein, 291 aa, Myxococcus fulvus
MKPHISILGAGRMGSALVKAFLQNEYTTTVWNRTRARCEPLAAAGARIADSVRDAVQTASVVIVNVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 3 - protein, 291 aa, Artificial Sequence
  Description of Artificial Sequence Synthetic polypeptide
MKPHISILGAGRMGSAAVKAFLQNEYTTTVWNRTRARCDPLAAAGARIADSVRDAVQTASVVIVNVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 4 - protein, 291 aa, Artificial Sequence
  Description of Artificial Sequence Synthetic polypeptide
MKPHISILGAGRMGSAAVKAFLQNEYTTTVWNRTRARCEPLAAAGARIADSVRDAVQTASVVIVNVFDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 5 - protein, 291 aa, Artificial Sequence
  Description of Artificial Sequence Synthetic polypeptide
MKPHISILGAGRMGSALTKAFAQNEYTTTVWNRTRARCEPLAAAGARIADSVRDAVQTASVVIVNVFDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 6 - protein, 291 aa, Artificial Sequence
  Description of Artificial Sequence Synthetic polypeptide
MKPHISILGAGRMGSALTKAFLQNEYTTTVWNRTRARCDPLAAAGARIADSVRDAVQTASVVIVNVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 7 - protein, 291 aa, Artificial Sequence
  Description of Artificial Sequence Synthetic polypeptide
MKPHISILGAGRMGSALVKAFAQNEYTTTVWNRTRARCEPLAAAGARIADSVRDAVQTASVVIENVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 8 - protein, 291 aa, Artificial Sequence
  Description of Artificial Sequence Synthetic polypeptide
MKPHISILGAGRMGSALVKAFAQNEYTTTVWNRTRARCEPLAAAGARIADSVRDAVQTASVVIENVNDYDTSDALLRQDEVTQELRGKVLVQTTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 9 - protein, 291 aa, Artificial Sequence
  Description of Artificial Sequence Synthetic polypeptide
MKPHISILGAGRMGSALVKAFLQNEYTTTVWNRTRARCAPLAAAGARIADSVRDAVQTASVVIVNVNDYDTSDALLRQDEVTQELRGKVLVQLTSGSPKLAREQATWARRHGIWYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 10 - protein, 291 aa, Artificial Sequence
  Description of Artificial Sequence Synthetic polypeptide
MKPHISILGAGRMGSALVKAFLQNEYTTTVWNRTRARCGPLAAAGARIADSVRDAVQTASVVIVNVNDYDTSDALLRQDEVTQELRGKVLVQTTSGSPKLAREQATWARRHGIWYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 11 - protein, 291 aa, Artificial Sequence
  Description of Artificial Sequence Synthetic polypeptide
MKPHISILGAGRMGSALVKAFLQNEYTTTVWNRTRARCEPLAAGGARIADSVRDAVQTASVVIVNVNDYDTSDALLRQDEVTQELRGKVLVQTTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 12 - protein, 291 aa, Artificial Sequence
  Description of Artificial Sequence Synthetic polypeptide
MKPHISILGAGRMGSALVKAFLQNEYTTTVWNRTRARCEPLAAGGARIADSVRDAVQTASVVIENVFDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 13 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIENVFDYDTSDALLRQDEVTQELRGKVLVQLTSGSPKLAREQATWARRHGIDYLDGAIMAWPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHNVAFQHLLHLCEERNIHRALPEAMDALIQTARKAGHGQDDFSVLARFLR

SEQ ID NO: 14 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIVNVFDYDTSDALLRQDEVTQELRGKVLVQKTSGSPKLAREQATWARRHGIDYLDGAIMAWPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 15 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIENVFDYDTSDALLRQDEVTQELRGKVLVQLTSGSPKLAREQATWARRHGIDYLDGAIMAFPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 16 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIVNVFDYDTSDALLRQDEVTQELRGKVLVQLTSGSPKLAREQATWARRHGIWYLDGAIMAFPDLIGRPDCTLLYAGPKALYDKHQAVLAALGGNTQHVSEDEGHASALDSAILFQLWGSLFSGLQAAAICRAEGIALDALGPHLEAVAAMIQFSMKDLLQRIQKEQFGADTESPATLDTHLVAFQHLLHLCEERNIHRALPEAMDALIQTARKAGHGQDDFSVLARFLR

SEQ ID NO: 17 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIVNVNDYDTSDALLRQDEVTQELRGKVLVQKTSGSPKLAREQATWARRHGIDYLDGAIMAFPDLIGRPDCTLLYAGPKALYDKHQAVLAALGGNTQHVSEDEGHASALDSAILFQLWGSLFSGLQAAAICRAEGIALDALGPHLEAVAASIQFSMKDLLQRIQKEQFGADTESPATLDTHLVAFQHLLHLCEERNIHRALPEAMDALIQTARKAGHGQDDFSVLARFLR

SEQ ID NO: 18 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIVNVNDYDTSDALLRQDEVTQELRGKVLVQKTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAASIQFSMKDLLQRIQKEQFGADTESPATLDTHLVAFQHLLHLCEERNIHRALPEAMDALIQTARKAGHGQDDFSVLARFLR

SEQ ID NO: 19 - protein, 291 aa, Artificial Sequence
  Description of Artificial Sequence Synthetic polypeptide
MKPHISILGAGRMGSALVKAFLQNEYTTTVWNRTRARCEPLAAAGARIADSVRDAVQTASVVIVNVNDYDTSDALLRQDEVTQELRGKVLVQLTSGSPKLAREQATWARRHGIDYLDGAIMATPDLIGRPDCTLLYAGPKALYDKHQAVLAALGGNTQHVSEDEGHASALDSAILFQLWGSLFSGLQAAAICRAEGIALDALGPHLEAVAASIQFSMKDLLQRIQKEQFGADTESPATLDTHLVAFQHLLHLCEERNIHRALPEAMDALIQTARKAGHGQDDFSVLARFLR

SEQ ID NO: 20 - DNA, 876 bp, Myxococcus fulvus
tcagcgcaggaagcgcgcgaggaccgagaagtcgtcctggccatgccccgccttccgggcggtctggatgagtgcatccatggcctcggggagagcgcggtggatgttgcgctcctcgcacaggtgcagcaggtgctggaacgccacgttgtgcgtgtcgagcgtggcggggctctcggtgtccgcgccgaactgctccttctggatgcgctggaggaggtccttcatgctgaactgaatcatggccgcgacggcctccagatgcgggccgagcgcgtcgagcgcaatcccctcggcgcggcagatggccgcggcctgcagcccgctgaacagcgaaccccacagctggaacaggatggcgctgtcgagcgcggacgcgtggccctcgtcctcgctcacgtgctgcgtgtttccaccgagcgccgccagcacggcctggtgcttgtcgtacagggccttcgggcccgcgtacaggagcgtgcagtcgggccggccgatgaggtccggcgtggccatgatggcgccgtccaggtagtcgatgccgtgccgtcgtgcccacgtcgcctgctcgcgcgccagcttcggtgagccggacgtgagctgcaccagcaccttgccccggagctcctgcgtcacctcgtcctggcgcagcagcgcgtcgctggtgtcgtagtcattcacgttcacgatgacgacgctggctgtctgcaccgcgtctcgcacggagtcggcgatgcgcgcgcccgctgccgccaacggctcgcaccgcgcccgggtgcgattccagaccgtggtcgtgtactcgttctggaggaacgccttgaccagcgcggagcccatgcggcccgcgccgaggatgctgatgtgtggcttcat

SEQ ID NO: 21 - DNA, 876 bp, Myxococcus fulvus
atgaagccacacatcagcatcctcggcgcgggccgcatgggctccgcgctggtcaaggcgttcctccagaacgagtacacgaccacggtctggaatcgcacccgggcgcggtgcgagccgttggcggcagcgggcgcgcgcatcgccgactccgtgcgagacgcggtgcagacagccagcgtcgtcatcgtgaacgtgaatgactacgacaccagcgacgcgctgctgcgccaggacgaggtgacgcaggagctccggggcaaggtgctggtgcagctcacgtccggctcaccgaagctggcgcgcgagcaggcgacgtgggcacgacggcacggcatcgactacctggacggcgccatcatggccacgccggacctcatcggccggcccgactgcacgctcctgtacgcgggcccgaaggccctgtacgacaagcaccaggccgtgctggcggcgctcggtggaaacacgcagcacgtgagcgaggacgagggccacgcgtccgcgctcgacagcgccatcctgttccagctgtggggttcgctgttcagcgggctgcaggccgcggccatctgccgcgccgaggggattgcgctcgacgcgctcggcccgcatctggaggccgtcgcggccatgattcagttcagcatgaaggacctcctccagcgcatccagaaggagcagttcggcgcggacaccgagagccccgccacgctcgacacgcacaacgtggcgttccagcacctgctgcacctgtgcgaggagcgcaacatccaccgcgctctccccgaggccatggatgcactcatccagaccgcccggaaggcggggcatggccaggacgacttctcggtcctcgcgcgcttcctgcgctga

SEQ ID NO: 22 - DNA, 873 bp, Artificial Sequence
  Description of Artificial Sequence Synthetic polynucleotide
atggccagtatttgtgtggtgggtgcaggtcgtatgggtagcgccctggcacgtgcatttctgcgcgcaggttatgtgacccatgtttggaatcgtaccccggcaaaaggtgaagccctggcagcactgggtgcccgttttgttccgagtctgcatcaggcaattgccgccagcgatattgttgttgtgaatgtgattgattacgccgccgccgatgcacatctgcgcagtgccagcgtgacccgcgccttaggtcgtaaactgctggtgcagctgaccagtggcagcccgagtcaggcccgccagaccggtgaatgggccaaaggtcatggcgtgggttatctggatggcgccattatggccaccccgaattttattggcgaaccgagtgcaaccattctgtatagcggtagtcagcatgcctttgatgaaaatcgcgatgtgtttctggcactgggcggtaatgccgttcatgttggtgacgattttggccatgccagcgccctggatattgccctgctgagccagctgtggggtaccctgtttggtaccctgcaggcaattgcggtgagccaggccgaaggtattgaactggatgcctatgcccgctatctgcagccgtttaaaccgaccattgatggtgccgttgccgatctggttacccgtgcccgtgatggtcgttatcgcggcgatgatcagaccctggcagcaattgccgcacattatagtgcctttcagccgctgctggaagttagccgcgaacgtggcctgaatcgcgcagtgccggatgcatttgatagtatttttaaagccgccattgccgcaggccatctgcaggatgattttgccgccctgacccgttttatgcgctaa

SEQ ID NO: 23 - protein, 290 aa, Bosea sp.
MASICVVGAGRMGSALARAFLRAGYVTHVWNRTPAKGEALAALGARFVPSLHQAIAASDIVVVNVIDYAAADAHLRSASVTRALGRKLLVQLTSGSPSQARQTGEWAKGHGVGYLDGAIMATPNFIGEPSATILYSGSQHAFDENRDVFLALGGNAVHVGDDFGHASALDIALLSQLWGTLFGTLQAIAVSQAEGIELDAYARYLQPFKPTIDGAVADLVTRARDGRYRGDDQTLAAIAAHYSAFQPLLEVSRERGLNRAVPDAFDSIFKAAIAAGHLQDDFAALTRFMR

(19) United States
(12) Patent Application Publication
(10) Pub. No.: US 2022/0154231 A1
(43) Pub. Date: May 19, 2022

(54) METHODS FOR PREPARING NICOTINE AND INTERMEDIATES THEREOF

(71) Applicants: Shandong Jincheng Pharmaceutical Chemical Co., Ltd, Shandong (CN); Shenzhen Hangsen Star Technology Co., Ltd, Shenzhen (CN)

(72) Inventors: Jiaquan LI, Zibo City (CN); Xianqiang MENG, Zibo City (CN); Genghui WEI, Zibo City (CN)

(21) Appl. No.: 17/380,777

(22) Filed: Jul. 20, 2021

(30) Foreign Application Priority Data
Nov. 18, 2020 (CN) 2020112954567
Dec. 14, 2020 (CN) 2020114670856

(51) Int. Cl. C12P 17/16; C12N 9/06; C12N 9/04; C07D 401/04

(52) U.S. Cl. CPC C12P 17/165; C12N 9/0028; C07D 401/04; C12Y 101/01047; C12N 9/0006

(57) ABSTRACT

Described are modified nucleic acids encoding an imine reductase enzyme. Also described are modified imine reductase enzymes. In some embodiments, the imine reductase enzymes may be used to produce products and intermediates thereof, such as (S)-nicotine.

### CROSS REFERENCE TO RELATED APPLICATIONS

This application claims priority to Chinese Patent Application No. 2020112954567, filed Nov. 18, 2020 and Chinese Patent Application No. 2020114670856, filed Dec. 14, 2020, the disclosures of which is incorporated herein by reference.

### TECHNICAL FIELD

The present disclosure relates to wild type and modified imine reductases and to methods for producing nicotine and intermediates thereof using imine reductase catalysis.

### SEQUENCE LISTING

The instant application contains a Sequence Listing which has been submitted electronically in ASCII format and is hereby incorporated by reference in its entirety. Said ASCII copy, created on Sep. 20, 2021, is named 120262_000004_SL_2.txt and is 55,307 bytes in size.

### BACKGROUND

Chiral amines and their derivatives are important branches of single enantiomer drugs and are structural units of many pharmaceutical intermediates and agrochemicals. At present, more than 70% of drugs are chiral amines and their derivatives, including neurological, antihypertensive, cardiovascular, and cerebral vascular drugs, and the like.

Optically pure 2-aryl(hetero) pyrrolidine is an important structural unit, and is commonly found in natural products, drug molecules, and synthetic intermediates. Functionalized chiral pyrrolidine compounds have recently been proven to have a variety of biological activities, especially suitable to serve as precursors for treating Parkinson's disease, Alzheimer's disease, and Tourette syndrome. In addition, many chiral 2-aryl(hetero)pyrrolidines are natural products and can be used as chiral bases, chiral auxiliaries, and chiral ligands.

The synthesis of chiral amines can be chemical or bioenzymatic. Chemical syntheses require a plurality of reactions, require stringent reagents, produce many impurities, often cannot achieve more than 98% of optical purity, are low-yielding, and are difficult to scale-up.

Enzymes commonly used in bioenzymatic catalysis of chiral amines mainly include transaminases, monoamine oxidases, dehydrogenases, and imine reductases, among others. However, substrate concentrations in a reduction process cannot be too high; otherwise the conversion rate is significantly reduced. To increase the substrate concentration, more expensive coenzymes, such as NAD(P)H, need to be added, resulting in higher costs.

Nicotine is a naturally occurring liquid alkaloid with strong physiological activity. Nicotine is commonly found mainly in natural tobacco and has important applications in agriculture, medical field, cosmetic industry, and the like. Further, nicotinic acid, nicotinamide, coramine, isoniazid, and other drugs can be prepared from nicotine through multiple-step reactions.

(S)-nicotine originates from plant extraction, but racemic nicotine can be obtained only through synthesis. The preparation of nicotine in the art is cumbersome. Many routes use expensive reagents, large amounts of organic solvents, include complicated steps, take a long time, require low temperatures, require separations and purifications that are complex, or are costly. In addition, nicotine prepared synthetically is racemic, which must be separated and purified to provide (S)-nicotine.

### SUMMARY

In certain embodiments, the disclosure provides nucleic acid molecules encoding an imine reductase of Myxococcus fulvus according to SEQ ID NO: 2. The nucleic acid molecules typically have a nucleotide sequence codon optimized for heterologous expression. In some embodiments, the nucleic acid molecule has a nucleotide sequence according to SEQ ID NO: 1.

In other embodiments, the disclosure provides nucleic acid molecules encoding mutant imine reductases derived from the imine reductase of Myxococcus fulvus. In some embodiments, the mutant imine reductases have an amino acid sequence according to any one of SEQ ID NOs: 3-19.

In further embodiments, the disclosure provides bacterial host cells comprising the nucleic acid molecules. In some embodiments, the bacterial host cells comprise nucleic acid molecules encoding any one of imine reductase having an amino acid sequence of any one of SEQ ID NOs: 2-19.

In yet other embodiments, the disclosure provides crude enzyme solutions comprising an imine reductase having an amino acid sequence of any one of SEQ ID NOs: 2-19.

In still further embodiments, the disclosure provides methods of producing an imine reductase of Myxococcus fulvus according to SEQ ID NO: 2, or an imine reductase having an amino acid sequence of any one of SEQ ID NOs: 3-19, in bacterial host cells

In some embodiments, the disclosure relates to methods for preparing nornicotine. The methods include contacting myosmine with the novel enzyme described herein.

In other embodiments, the disclosure relates to methods for preparing nicotine.

In further embodiments, the disclosure relates to (S)-nicotine prepared according to any one of the methods described herein.

Other aspects and embodiments of the invention will be readily apparent from the following detailed description of the invention.

### DETAILED DESCRIPTION

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Imine Reductases

In certain aspects, the disclosure provides nucleotide and amino acid sequences for imine reductases derived from imine reductase (NAD(P)-dependent oxidoreductase) of Myxococcus fulvus. The imine reductases derived from imine reductase of Myxococcus fulvus may include the same amino acid sequence as the imine reductase of Myxococcus fulvus, but encoded by a nucleic acid molecule with a nucleotide sequence having one or more nucleotide substitutions.

The imine reductases may be derived from the imine reductase of the Myxococcus fulvus strain DSM 16525. The genomic sequence of Myxococcus fulvus strain DSM 16525 is available under accession number NZ_FOIB01000013. The NZ_FOIB01000013 Region: 55611..56486, negative strand, has a nucleotide sequence as follows (SEQ ID NO: 20):

(SEQ ID NO: 20)
TCAGCGCAGGAAGCGCGCGAGGACCGAGAAGTCGTCCTGGCCATGCCCC
GCCTTCCGGGCGGTCTGGATGAGTGCATCCATGGCCTCGGGGAGAGCGC
GGTGGATGTTGCGCTCCTCGCACAGGTGCAGCAGGTGCTGGAACGCCAC
GTTGTGCGTGTCGAGCGTGGCGGGGCTCTCGGTGTCCGCGCCGAACTGC
TCCTTCTGGATGCGCTGGAGGAGGTCCTTCATGCTGAACTGAATCATGG
CCGCGACGGCCTCCAGATGCGGGCCGAGCGCGTCGAGCGCAATCCCCTC
GGCGCGGCAGATGGCCGCGGCCTGCAGCCCGCTGAACAGCGAACCCCAC
AGCTGGAACAGGATGGCGCTGTCGAGCGCGGACGCGTGGCCCTCGTCCT
CGCTCACGTGCTGCGTGTTTCCACCGAGCGCCGCCAGCACGGCCTGGTG
CTTGTCGTACAGGGCCTTCGGGCCCGCGTACAGGAGCGTGCAGTCGGGC
CGGCCGATGAGGTCCGGCGTGGCCATGATGGCGCCGTCCAGGTAGTCGA
TGCCGTGCCGTCGTGCCCACGTCGCCTGCTCGCGCGCCAGCTTCGGTGA
GCCGGACGTGAGCTGCACCAGCACCTTGCCCCGGAGCTCCTGCGTCACC
TCGTCCTGGCGCAGCAGCGCGTCGCTGGTGTCGTAGTCATTCACGTTCA
CGATGACGACGCTGGCTGTCTGCACCGCGTCTCGCACGGAGTCGGCGAT
GCGCGCGCCCGCTGCCGCCAACGGCTCGCACCGCGCCCGGGTGCGATTC
CAGACCGTGGTCGTGTACTCGTTCTGGAGGAACGCCTTGACCAGCGCGG
AGCCCATGCGGCCCGCGCCGAGGATGCTGATGTGTGGCTTCAT

The reverse complement nucleotide sequence of SEQ ID NO: 20 is as follows (SEQ ID NO: 21):

(SEQ ID NO: 21)
ATGAAGCCACACATCAGCATCCTCGGCGCGGGCCGCATGGGCTCCGCGC
TGGTCAAGGCGTTCCTCCAGAACGAGTACACGACCACGGTCTGGAATCG
CACCCGGGCGCGGTGCGAGCCGTTGGCGGCAGCGGGCGCGCGCATCGCC
GACTCCGTGCGAGACGCGGTGCAGACAGCCAGCGTCGTCATCGTGAACG
TGAATGACTACGACACCAGCGACGCGCTGCTGCGCCAGGACGAGGTGAC
GCAGGAGCTCCGGGGCAAGGTGCTGGTGCAGCTCACGTCCGGCTCACCG
AAGCTGGCGCGCGAGCAGGCGACGTGGGCACGACGGCACGGCATCGACT
ACCTGGACGGCGCCATCATGGCCACGCCGGACCTCATCGGCCGGCCCGA
CTGCACGCTCCTGTACGCGGGCCCGAAGGCCCTGTACGACAAGCACCAG
GCCGTGCTGGCGGCGCTCGGTGGAAACACGCAGCACGTGAGCGAGGACG
AGGGCCACGCGTCCGCGCTCGACAGCGCCATCCTGTTCCAGCTGTGGGG
TTCGCTGTTCAGCGGGCTGCAGGCCGCGGCCATCTGCCGCGCCGAGGGG
ATTGCGCTCGACGCGCTCGGCCCGCATCTGGAGGCCGTCGCGGCCATGA
TTCAGTTCAGCATGAAGGACCTCCTCCAGCGCATCCAGAAGGAGCAGTT
CGGCGCGGACACCGAGAGCCCCGCCACGCTCGACACGCACAACGTGGCG
TTCCAGCACCTGCTGCACCTGTGCGAGGAGCGCAACATCCACCGCGCTC
TCCCCGAGGCCATGGATGCACTCATCCAGACCGCCCGGAAGGCGGGGCA
TGGCCAGGACGACTTCTCGGTCCTCGCGCGCTTCCTGCGCTGA

SEQ ID NO: 21 codes for the wild type imine reductase of Myxococcus fulvus (protein ID WP_074958336.1, SEQ ID NO: 2):

(SEQ ID NO: 2)
MKPHISILGAGRMGSALVKAFLQNEYTTTVWNRTRARCEPLAAAGARIA
DSVRDAVQTASVVIVNVNDYDTSDALLRQDEVTQELRGKVLVQLTSGSP
KLAREQATWARRHGIDYLDGAIMATPDLIGRPDCTLLYAGPKALYDKHQ
AVLAALGGNTQHVSEDEGHASALDSAILFQLWGSLFSGLQAAAICRAEG
IALDALGPHLEAVAAMIQFSMKDLLQRIQKEQFGADTESPATLDTHNVA
FQHLLHLCEERNIHRALPEAMDALIQTARKAGHGQDDFSVLARFLR

SEQ ID NO: 21 may be codon optimized for heterologous expression in host bacterial cells other than Myxococcus fulvus.

The codon optimized nucleotide sequence of SEQ ID NO: 21 typically has one or more nucleic acid substitutions that do not alter the amino acid sequence of the encoded protein. In some embodiments, the codon optimized nucleotide sequence of SEQ ID NO: 21 has 1, 2, 3, 4, 5, 6, 7, 8, 9, 10, or more nucleic acid substitutions which do not alter the amino acid sequence of the encoded protein. In some embodiments, the codon optimized nucleotide sequence of SEQ ID NO: 21 has between 1 and 10 nucleic acid substitutions which do not alter the amino acid sequence of the encoded protein. In some embodiments, the codon optimized nucleotide sequence of SEQ ID NO: 21 has between 1 and 20 nucleic acid substitutions which do not alter the amino acid sequence of the encoded protein. In some embodiments, the codon optimized nucleotide sequence of SEQ ID NO: 21 has between 1 and 30 nucleic acid substitutions which do not alter the amino acid sequence of the encoded protein. In some embodiments, the codon optimized nucleotide sequence of SEQ ID NO: 21 has between 1 and 40 nucleic acid substitutions which do not alter the amino acid sequence of the encoded protein. In some embodiments, the codon optimized nucleotide sequence of SEQ ID NO: 21 has between 1 and 50 nucleic acid substitutions which do not alter the amino acid sequence of the encoded protein. In some embodiments, the codon optimized nucleotide sequence of SEQ ID NO: 21 has between 1 and 60 nucleic acid substitutions which do not alter the amino acid sequence of the encoded protein. In some embodiments, the codon optimized nucleotide sequence of SEQ ID NO: 21 has between 1 and 70 nucleic acid substitutions which do not alter the amino acid sequence of the encoded protein. In some embodiments, the codon optimized nucleotide sequence of SEQ ID NO: 21 has between 1 and 80 nucleic acid substitutions which do not alter the amino acid sequence of the encoded protein. In some embodiments, the codon optimized nucleotide sequence of SEQ ID NO: 21 has between 1 and 90 nucleic acid substitutions which do not alter the amino acid sequence of the encoded protein. In some embodiments, the codon optimized nucleotide sequence of SEQ ID NO: 21 has between 1 and 100 nucleic acid substitutions which do not alter the amino acid sequence of the encoded protein. In some embodiments, the codon optimized nucleotide sequence of SEQ ID NO: 21 has between 1 and 110 nucleic acid substitutions which do not alter the amino acid sequence of the encoded protein. In some embodiments, the codon optimized nucleotide sequence of SEQ ID NO: 21 has between 1 and 120 nucleic acid substitutions which do not alter the amino acid sequence of the encoded protein. In some embodiments, the codon optimized nucleotide sequence of SEQ ID NO: 21 has between 1 and 130 nucleic acid substitutions which do not alter the amino acid sequence of the encoded protein. In some embodiments, the codon optimized nucleotide sequence of SEQ ID NO: 21 has between 1 and 140 nucleic acid substitutions which do not alter the amino acid sequence of the encoded protein. In some embodiments, the codon optimized nucleotide sequence of SEQ ID NO: 21 has between 1 and 150 nucleic acid substitutions which do not alter the amino acid sequence of the encoded protein. In some embodiments, the codon optimized nucleotide sequence of SEQ ID NO: 21 has between 1 and 200 nucleic acid substitutions which do not alter the amino acid sequence of the encoded protein.

For example, the codon optimized nucleotide sequence of SEQ ID NO: 21 is SEQ ID NO: 1 shown below:

(SEQ ID NO: 1)
ATGAAGCCGCATATTAGTATTCTGGGTGCAGGTCGTATGGGCAGTGCCC
TGGTGAAAGCATTTCTGCAGAATGAATATACCACCACCGTTTGGAATCG
TACCCGTGCACGTTGTGAACCGCTGGCAGCAGCAGGCGCCCGTATTGCC
GATAGCGTTCGCGATGCAGTGCAGACCGCCAGCGTGGTTATTGTGAATG
TGAATGATTATGATACCAGCGATGCCCTGCTGCGCCAGGATGAAGTGAC
CCAGGAACTGCGCGGTAAAGTTCTGGTGCAGCTGACCAGCGGCAGTCCG
AAACTGGCCCGCGAACAGGCCACCTGGGCCAGACGTCATGGTATTGATT
ATCTGGATGGTGCAATTATGGCCACCCCGGATCTGATTGGTCGTCCGGA
TTGTACCCTGCTGTATGCCGGCCCGAAAGCACTGTATGATAAACATCAG
GCCGTTCTGGCAGCACTGGGTGGCAATACCCAGCATGTTAGTGAAGATG
AAGGTCATGCAAGCGCACTGGATAGTGCCATTCTGTTTCAGCTGTGGGG
TAGCCTGTTTAGTGGTCTGCAGGCCGCCGCAATTTGTCGTGCAGAAGGC
ATTGCCCTGGATGCACTGGGTCCGCATCTGGAAGCAGTGGCCGCCATGA
TTCAGTTTAGCATGAAAGATCTGCTGCAGCGTATTCAGAAAGAACAGTT
TGGTGCAGATACCGAAAGCCCGGCAACCCTGGATACCCATAATGTTGCC
TTTCAGCATCTGCTGCATCTGTGCGAAGAACGTAATATTCATCGCGCCC
TGCCGGAAGCAATGGATGCACTGATTCAGACCGCACGCAAAGCCGGTCA
TGGCCAGGATGATTTTAGTGTTCTGGCACGTTTTCTGCGTTAA

SEQ ID NO: 21 and SEQ ID NO: 1 encode SEQ ID NO: 2. However, the nucleotide sequence of SEQ ID NO: 1 includes one or more nucleotide substitutions, such as between 1 and about 150 or more nucleotide substitutions, relative to SEQ ID NO: 21:

| | | |
|---|---|---|
| SEQ_ID_NO_21 | ATGAAGCCACAGATCAGCATCCTCGGCGCGGGCCGCATGGGCTCCGCGCTGGTCAAGGCG | 60 |
| SEQ_ID_NO_1 | ATGAAGCCGCATATTAGTATTCTGGGTGCAGGTCGTATGGGCAGTGCCCTGGTGAAAGCA | 60 |
| | ******** ** ** ** ** ** ** ** ** ** ****** ** ***** ** ** | |
| SEQ_ID_NO_21 | TTCCTCCAGAACGAGTACACGACCACGGTCTGGAATCGCACCCGGGCGCGGTGCGAGGCG | 120 |
| SEQ_ID_NO_l | TTTCTGCAGAATGAATATACCACCACCGTTTGGAATCGTACCCGTGCACGTTGTGAACCG | 120 |
| | ** ** ***** ** ** ** ***** ** ******** ***** ** ** ** ** *** | |
| SEQ_ID_NO_21 | TTGGCGGCAGCGGGCGCGCGCATCGCCGACTCCGTGCGAGACGCGGTGCAGACAGCCAGC | 180 |
| SEQ_ID_NO_1 | CTGGCAGCAGCAGGCGCCCGTATTGCCGATAGCGTTCGCGATGCAGTGCAGACCGCCAGC | 180 |
| | **** ***** ***** ** ** ***** *** ** ** ** ******** ****** | |
| SEQ_ID_NO_21 | GTCGTCATCGTGAACGTGAATGACTACGACACCAGCGACGCGCTGCTGCGCCAGGACGAG | 240 |
| SEQ_ID_NO_1 | GTGGTTATTGTGAATGTGAATGATTATGATACCAGCGATGCCCTGCTGCGCCAGGATGAA | 240 |
| | ** ** ** ***** ******** ** ** ******** ** ************** ** | |
| SEQ_ID_NO_21 | GTGACGCAGGAGCTCCGGGGCAAGGTGCTGGTGCAGCTCACGTCCGGCTCACCGAAGCTG | 300 |
| SEQ_ID_NO_1 | GTGACCCAGGAACTGCGCGGTAAAGTTCTGGTGCAGCTGACCAGCGGCAGTCCGAAACTG | 300 |
| | ***** ***** ** ** ** ** ** *********** ** **** ***** *** | |
| SEQ_ID_NO_21 | GCGCGCGAGCAGGCGACGTGGGCACGACGGCACGGCATCGACTACCTGGACGGCGCCATC | 360 |
| SEQ_ID_NO_ | GCCCGCGAACAGGCCACCTGGGCCAGACGTCATGGTATTGATTATCTGGATGGTGCAATT | 360 |
| | ** ***** ***** ** ***** **** ** ** ** ** ** ***** ** ** ** | |
| SEQ_ID_NO_21 | ATGGCCACGCCGGACCTCATCGGCCGGCCCGACTGCACGCTCCTGTACGCGGGCCCGAAG | 420 |
| SEQ_ID_NO_1 | ATGGCCACCCCGGATCTGATTGGTCGTCCGGATTGTACCCTGCTGTATGCCGGCCCGAAA | 420 |
| | ******** ***** ** ** ** ** ** ** ** ** ** ***** ** ******** | |
| SEQ_ID_NO_21 | GCCCTGTACGACAAGCACCAGGCCGTGCTGGCGGCGCTCGGTGGAAACACGCAGCACGTG | 480 |
| SEQ_ID_NO_1 | GCACTGTATGATAAACATCAGGCCGTTCTGGCAGCACTGGGTGGCAATACCCAGCATGTT | 480 |
| | ** ***** ** ** ** ******** ***** ** ** ***** ** ** ***** ** | |
| SEQ_ID_NO_21 | AGCGAGGACGAGGGCCACGCGTCCGCGCTCGACAGCGCCATCCTGTTCCAGCTGTGGGGT | 540 |
| SEQ_ID_NO_1 | AGTGAAGATGAAGGTCATGCAAGCGCACTGGATAGTGCCATTCTGTTTCAGCTGTGGGGT | 540 |
| | ** ** ** ** ** ** ** *** ** ** ** ***** ***** ************ | |
| SEQ_ID_NO_21 | TCGCTGTTCAGCGGGCTGCAGGCCGCGGCCATCTGCCGCGCCGAGGGGATTGCGCTCGAC | 600 |
| SEQ_ID_NO_1 | AGCCTGTTTAGTGGTCTGCAGGCCGCCGCAATTTGTCGTGCAGAAGGCATTGCCCTGGAT | 600 |
| | ***** ** ** *********** ** ** ** ** ** ** ** ***** ** ** | |
| SEQ_ID_NO_21 | GCGCTCGGCCCGCATCTGGAGGCCGTCGCGGCCATGATTCAGTTCAGCATGAAGGACCTC | 660 |
| SEQ_ID_NO_1 | GCACTGGGTCCGCATCTGGAAGCAGTGGCCGCCATGATTCAGTTTAGCATGAAAGATCTG | 660 |
| | ** ** ** *********** ** ** ** ************** ******** ** ** | |
| SEQ_ID_NO_21 | CTCCAGCGCATCCAGAAGGAGCAGTTCGGCGCGGACACCGAGAGCCCCGCCACGCTCGAC | 720 |
| SEQ_ID_NO_1 | CTGCAGCGTATTCAGAAAGAACAGTTTGGTGCAGATACCGAAAGCCCGGCAACCCTGGAT | 720 |
| | ** ***** ** ***** ** ***** ** ** ** ***** ***** ** ** ** ** | |
| SEQ_ID_NO_21 | ACGCACAACGTGGCGTTCCAGCACCTGCTGCACCTGTGCGAGGAGCGCAACATCCACCGC | 780 |
| SEQ_ID_NO_l | ACCCATAATGTTGCCTTTCAGCATCTGCTGCATCTGTGCGAAGAACGTAATATTCATCGC | 780 |
| | ** ** ** ** ** ** ***** ******** ******** ** ** ** ** ** *** | |
| SEQ_ID_NO_21 | GCTCTCCCCGAGGCCATGGATGCACTCATCCAGACCGCCCGGAAGGCGGGGCATGGCCAG | 840 |
| SEQ_ID_NO_l | GCCCTGCCGGAAGCAATGGATGCACTGATTCAGACCGCACGCAAAGCCGGTCATGGCCAG | 840 |
| | ** ** ** ** ** *********** ** ******** ** ** ** ** ********* | |
| SEQ_ID_NO_21 | GACGACTTCTCGGTCCTCGCGCGCTTCCTGCGCTGA | 876 |
| SEQ_ID_NC_1 | GATGATTTTAGTGTTCTGGCACGTTTTCTGCGTTAA | 876 |
| | ** ** ** ** ** ** ** ** ***** * * | |

In some embodiments, the nucleic acid molecule encoding an imine reductase of Myxococcus fulvus according to SEQ ID NO: 2 has a nucleotide sequence codon optimized for heterologous expression. In some embodiments, the nucleic acid molecule encoding an imine reductase of Myxococcus fulvus according to SEQ ID NO: 2 has a nucleotide sequence according to SEQ ID NO: 21 and codon optimized for heterologous expression. In some embodiments, the nucleic acid molecule encoding an imine reductase of Myxococcus fulvus according to SEQ ID NO: 2 has a nucleotide sequence according to SEQ ID NO: 21 and one or more nucleotide substitutions from codon optimization for heterologous expression. Codon optimization methods are known in the art and utilize codon usage bias of the host organism to change the codons in the target nucleic acid (Puigbo et al., Nucleic Acids Research, 35, Web Server issue, W126-W131 (2007)).

In some embodiments, the nucleic acid molecule encoding an imine reductase of Myxococcus fulvus according to SEQ ID NO: 2 has a substantially similar nucleic acid sequence to SEQ ID NO21. In some embodiments, the nucleic acid molecule encoding an imine reductase of Myxococcus fulvus according to SEQ ID NO: 2 has at least about 70% sequence identity with SEQ ID NO: 21. In some embodiments, the nucleic acid molecule encoding an imine reductase of Myxococcus fulvus according to SEQ ID NO: 2 has at least about 75% sequence identity with SEQ ID NO: 21. In some embodiments, the nucleic acid molecule encoding an imine reductase of Myxococcus fulvus according to SEQ ID NO: 2 has at least about 80% sequence identity with SEQ ID NO: 21. In some embodiments, the nucleic acid molecule encoding an imine reductase of Myxococcus fulvus according to SEQ ID NO: 2 has at least about 85% sequence identity with SEQ ID NO: 21. In some embodiments, the nucleic acid molecule encoding an imine reductase of Myxococcus fulvus according to SEQ ID NO: 2 has at least about 90% sequence identity with SEQ ID NO: 21. In some embodiments, the nucleic acid molecule encoding an imine reductase of Myxococcus fulvus according to SEQ ID NO: 2 has at least about 85% sequence identity with SEQ ID NO: 21.

Substantially similar” with respect to nucleic acid or amino acid sequences means at least about 65% sequence identity between two or more sequences. The term refers to at least about 70% sequence identity between two or more sequences, at least about 75% sequence identity, at least about 80% sequence identity, at least about 85% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, or at least about 99% or greater sequence identity. Such identity can be determined using algorithms known in the art, such as the mBLAST algorithm.

The nucleic acid molecule encoding an imine reductase of Myxococcus fulvus according to SEQ ID NO: 2 may have a nucleotide sequence according to SEQ ID NO: 1.

Similar nucleotide sequence codon optimization may be conducted on the coding region for the imine reductase (NAD(P)-dependent oxidoreductase) of Bosea sp., such as Bosea sp. BIWAKO-01. An exemplary codon optimized nucleotide sequence encoding the imine reductase (NAD(P)-dependent oxidoreductase) of Bosea sp. is shown below (SEQ ID NO: 22):

(SEQ ID NO: 22)
ATGGCCAGTATTTGTGTGGTGGGTGCAGGTCGTATGGGTAGCGCCCTGG
CACGTGCATTTCTGCGCGCAGGTTATGTGACCCATGTTTGGAATCGTAC
CCCGGCAAAAGGTGAAGCCCTGGCAGCACTGGGTGCCCGTTTTGTTCCG
AGTCTGCATCAGGCAATTGCCGCCAGCGATATTGTTGTTGTGAATGTGA
TTGATTACGCCGCCGCCGATGCACATCTGCGCAGTGCCAGCGTGACCCG
CGCCTTAGGTCGTAAACTGCTGGTGCAGCTGACCAGTGGCAGCCCGAGT
CAGGCCCGCCAGACCGGTGAATGGGCCAAAGGTCATGGCGTGGGTTATC
TGGATGGCGCCATTATGGCCACCCCGAATTTTATTGGCGAACCGAGTGC
AACCATTCTGTATAGCGGTAGTCAGCATGCCTTTGATGAAAATCGCGAT
GTGTTTCTGGCACTGGGCGGTAATGCCGTTCATGTTGGTGACGATTTTG
GCCATGCCAGCGCCCTGGATATTGCCCTGCTGAGCCAGCTGTGGGGTAC
CCTGTTTGGTACCCTGCAGGCAATTGCGGTGAGCCAGGCCGAAGGTATT
GAACTGGATGCCTATGCCCGCTATCTGCAGCCGTTTAAACCGACCATTG
ATGGTGCCGTTGCCGATCTGGTTACCCGTGCCCGTGATGGTCGTTATCG
CGGCGATGATCAGACCCTGGCAGCAATTGCCGCACATTATAGTGCCTTT
CAGCCGCTGCTGGAAGTTAGCCGCGAACGTGGCCTGAATCGCGCAGTGC
CGGATGCATTTGATAGTATTTTTAAAGCCGCCATTGCCGCAGGCCATCT
GCAGGATGATTTTGCCGCCCTGACCCGTTTTATGCGCTAA

SEQ ID NO: 22 encodes the imine reductase of Bosea sp. (protein ID: WP_069881969.1), having amino acid sequence as shown below (SEQ ID NO: 23):

(SEQ ID NO: 23)
MASICVVGAGRMGSALARAFLRAGYVTHVWNRTPAKGEALAALGARFVP
SLHQAIAASDIVVVNVIDYAAADAHLRSASVTRALGRKLLVQLTSGSPS
QARQTGEWAKGHGVGYLDGAIMATPNFIGEPSATILYSGSQHAFDENRD
VFLALGGNAVHVGDDFGHASALDIALLSQLWGTLFGTLQAIAVSQAEGI
ELDAYARYLQPFKPTIDGAVADLVTRARDGRYRGDDQTLAAIAAHYSAF
QPLLEVSRERGLNRAVPDAFDSIFKAAIAAGHLQDDFAALTRFMR.

Mutant Imine Reductases

The disclosure also provides one or more mutant imine reductases derived from the imine reductase of Myxococcus fulvus. The amino acid sequences for the one or more mutant imine reductases derived from the imine reductase of Myxococcus fulvus may be substantially similar to the amino acid sequence of SEQ ID NO: 2. In some embodiments, the mutant imine reductases have at least about 80% amino acid sequence identity with SEQ ID NO: 2. In some embodiments, the mutant imine reductases have at least about 85% amino acid sequence identity with SEQ ID NO: 2. In some embodiments, the mutant imine reductases have at least about 90% amino acid sequence identity with SEQ ID NO: 2. In some embodiments, the mutant imine reductases have at least about 95% amino acid sequence identity with SEQ ID NO: 2. In some embodiments, the mutant imine reductases have between one and ten amino acid substitutions relative to the amino acid sequence of SEQ ID NO: 2. In some embodiments, the mutant imine reductases have between one and eight amino acid substitutions relative to the amino acid sequence of SEQ ID NO: 2. In some embodiments, the mutant imine reductases have between one and six amino acid substitutions relative to the amino acid sequence of SEQ ID NO: 2. In some embodiments, the mutant imine reductases have two amino acid substitutions relative to the amino acid sequence of SEQ ID NO: 2. In some embodiments, the mutant imine reductases have three amino acid substitutions relative to the amino acid sequence of SEQ ID NO: 2. In some embodiments, the mutant imine reductases have four amino acid substitutions relative to the amino acid sequence of SEQ ID NO: 2. In some embodiments, the mutant imine reductases have five amino acid substitutions relative to the amino acid sequence of SEQ ID NO: 2.

The amino acid sequences for the one or more mutant imine reductases derived from the imine reductase of Myxococcus fulvus are described below and include SEQ ID NOs: 3-19.

SEQ ID NO: 3 corresponds to the amino acid sequence shown in SEQ ID NO: 2 except that leucine at site 17 is replaced by alanine and glutamic acid at site 39 is replaced by aspartic acid.

SEQ ID NO: 4 corresponds to the amino acid sequence shown in SEQ ID NO: 2 except that leucine at site 17 is replaced by alanine and asparagine at position 67 is replaced by phenylalanine.

SEQ ID NO: 5 corresponds to the amino acid sequence as shown in SEQ ID NO: 2 except that valine at position 18 is replaced by threonine, proline at position 22 is replaced by alanine, and asparagine at position 67 is replaced by phenylalanine.

SEQ ID NO: 6 corresponds to the amino acid sequence shown in SEQ ID NO: 2 except that valine at position 18 is replaced by threonine, glutamic acid at site 39 is replaced by aspartic acid; and asparagine at position 243 is replaced by leucine.

SEQ ID NO: 7 corresponds to the amino acid sequence as shown in SEQ ID NO: 2 except that proline at position 22 is replaced by alanine, valine at position 64 is replaced by glutamic acid; and asparagine at position 243 is replaced by leucine.

SEQ ID NO: 8 corresponds to the amino acid sequence as shown in SEQ ID NO: 2 except that proline at position 22 is replaced by alanine, valine at position 64 is replaced by glutamic acid, and leucine at position 93 is replaced by threonine.

SEQ ID NO: 9 corresponds to the amino acid sequence as shown in SEQ ID NO: 2 except that glutamic acid at position 39 is replaced by alanine, and aspartic acid at position 114 is replaced by tryptophan.

SEQ ID NO: 10 corresponds to the amino acid sequence shown in SEQ ID NO: 2 except that glutamic acid at position 39 is replaced by glycine, leucine at position 93 is replaced by threonine, aspartic acid at position 114 is replaced by tryptophan; and asparagine at position 243 is replaced by leucine.

SEQ ID NO: 11 corresponds to the amino acid sequence as shown in SEQ ID NO: 2 except that alanine at position 44 is replaced by glycine, leucine at the position 93 is replaced by threonine, and asparagine at position 243 is replaced by leucine.

SEQ ID NO: 12 corresponds to the amino acid sequence as shown in SEQ ID NO: 2 except that alanine at position 44 is replaced by glycine, valine at position 64 is replaced by glutamic acid, asparagine at position 67 is replaced by phenylalanine, and asparagine at position 243 is replaced by leucine.

SEQ ID NO: 13 corresponds to the amino acid sequence as shown in SEQ ID NO: 2 except that valine at the position 64 is replaced by glutamic acid, asparagine at position 67 is replaced by phenylalanine, and threonine at position 123 is replaced by tryptophan.

SEQ ID NO: 14 corresponds to the amino acid sequence as shown in SEQ ID NO: 2 except that asparagine at position 67 is replaced by phenylalanine, leucine at position 93 is replaced by lysine, threonine at position 123 is replaced by tryptophan, and asparagine at position 243 is replaced by leucine.

SEQ ID NO: 15 corresponds to the amino acid sequence as shown in SEQ ID NO: 2 except that valine at the position 64 is replaced by glutamic acid, asparagine at position 67 is replaced by phenylalanine, threonine at position 123 is replaced by phenylalanine, and asparagine at position 243 is replaced by leucine.

SEQ ID NO: 16 corresponds to the amino acid sequence as shown in SEQ ID NO: 2 except that asparagine at position 67 is replaced by phenylalanine, aspartic acid at the position 114 is replaced by tryptophan, threonine at position 123 is replaced by phenylalanine, and asparagine at position 243 is replaced by leucine.

SEQ ID NO: 17 corresponds to the amino acid sequence as shown in SEQ ID NO: 2 except that leucine at position 93 is replaced by lysine, threonine at position 123 is replaced by phenylalanine, methionine at position 212 is replaced by serine, and asparagine at position 243 is replaced by leucine.

SEQ ID NO: 18 corresponds to the amino acid sequence as shown in SEQ ID NO: 2 except that leucine at position 93 is replaced by lysine, methionine at position 212 is replaced by serine, and asparagine at position 243 is replaced by leucine.

SEQ ID NO: 19 corresponds to the amino acid sequence as shown in SEQ ID NO: 2 except that methionine at position 212 is replaced by serine and asparagine at position 243 is replaced by leucine.

Also described are nucleic acid molecules encoding the one or more mutant imine reductases derived from the imine reductase of Myxococcus fulvus. The nucleic acid molecules may include SEQ ID NO: 1 having one or more nucleotide substitutions to change the coded amino acid sequence to that of any one of SEQ ID NOs: 3-19.

Expression Vectors, Bacterial Host Cells, and Crude Enzyme Solution

The disclosure further provides expression vectors, bacterial host cells, and crude enzyme solution for production of the imine reductases derived from the imine reductase of Myxococcus fulvus.

Typically, the expression vector includes a nucleic acid molecule encoding an imine reductase having an amino acid sequence according to any one of SEQ ID NOs: 2-19. The expression vector may be a bacterial expression vector. An exemplary expression vector is a pET series plasmid, a pTXB series plasmid, a pGEX series plasmid, a pETduet series plasmid, a pTYB series plasmid, a pRSF series plasmid, a pET-28a(+) plasmid, or a pRSFDuet1 plasmid.

In some embodiments, the expression vector is a pET series plasmid. In some embodiments, the expression vector is a pTXB series plasmid. In some embodiments, the expression vector is a pGEX series plasmid. In some embodiments, the expression vector is a pETduet series plasmid. In some embodiments, the expression vector is a pTYB series plasmid. In some embodiments, the expression vector is a pRSF series plasmid.

In some embodiments, the expression vector is a pET-28a(+) plasmid.

In some embodiments, the expression vector is a dual expression vector having a nucleic acid molecule encoding an imine reductase having an amino acid sequence according to any one of SEQ ID NOs: 2-19 and a nucleic acid molecule encoding a glucose dehydrogenase. Dual expression vectors are known plasmids from a pRSF series or from a pETduet series. In some embodiments, the expression vector is pRSFDuet1 plasmid.

Exemplary host bacterial cells include Escherichia coli strains. Exemplary Escherichia coli strains that can host the expression of the nucleic acids may be expression strains BL21, ROSETTA™, ORIGAMI™, and TUNER™ strains.

Following expression of the nucleic acids, such as following fermenting, concentrating, resuspending of the cells in a buffer, the cells may be used to form a crude enzyme solution. The cells in the buffer may be broken down by a high-pressure homogenizer to obtain the crude enzyme solution used in the catalytic reaction.

### Methods of Preparing the Imine Reductases

The methods for preparing the genetically engineered bacteria that produce imine reductases include codon optimizing the gene of Myxococcus fulvus encoding MsIR1 WP_074958336.1, SEQ ID NO: 2, performing total synthesis on corresponding sequences, adding restriction enzyme cutting sites Nde I and EcoR I to both ends of the gene, inserting the synthesized gene into a corresponding expression vector, transforming the expression vector into a recipient bacterium to obtain the genetically engineered bacteria M1 producing imine reductases, and fermenting and culturing the genetically engineered bacteria to achieve efficient heterologous expression of imine reductases. The amino acid sequence encoded by the codon optimized MsIR1 gene of Myxococcus fulvus (SEQ ID NO: 1) is provided in SEQ ID NO: 2. The term “recombinant” and “genetically engineered” as used herein refer to bacteria that are not naturally occurring and are prepared in a laboratory setting.

Suitable host bacterium that are capable of efficiently expressing an exogenous gene for use in the preparation of the imine reductase-producing genetically engineered bacteria described herein include BL21, ROSETTA™, ORIGAMI™, or TUNER™ bacteria. In some embodiments, the host bacterium is BL21. In other embodiments, the host bacterium ROSETTA™. In further embodiments, the host bacterium is ORIGAMI™. In yet other embodiments, the host bacterium is TUNER™.

The transformation step is performed using a host with a plasmid that can grow based on known information and that can produce the imine reductases in the present disclosure. Any artificial or natural medium containing suitable carbon sources, nitrogen sources, and inorganic and other nutrients can be used provided that it can satisfy the growth of the host bacterium and allows for expression of a target protein. Culture methods and culture conditions are not limited, and can be appropriately selected based on a culture method, type, and the like, provided that the growth of the host can be satisfied and the corresponding active imine reductases can be produced.

The imine reductases used herein may be obtained from a culture of the foregoing genetically engineered recombinant bacteria, bacterial cells that are obtained by centrifuging a culture medium, or processed product of bacterial cells. The term “processed product” refers to an extract or a breakage solution of bacteria, a separated product obtained through separation and/or purification of the imine reductases, or an immobilized product obtained through immobilization of an extract or a processed product.

### Methods for Using Imine Reductases

The present disclosure also relates to methods for synthesizing a chiral 2-(3-pyridyl)-pyrrolidine compound through whole-cell or crude enzyme solution transformation. The methods comprise catalyzing a 2-pyridyl-1-pyrroline compound with imine reductases to obtain (S)-2-(3-pyridyl)-pyrrolidine.

The methods for preparing 2-pyridyl pyrrolidine compounds, such as (S)-2-(3-pyridyl)-pyrrolidine, include using imine reductases (IREDs) or engineered bacteria that express the enzymes and regenerate coenzymes from a glucose dehydrogenase/glucose system. In some embodiments, the disclosure provides methods for producing (S)-2-(3-pyridyl)-pyrrolidine by using imine reductases derived from Myxococcus as a biocatalyst and NADP(H) as a coenzyme from a glucose dehydrogenase/glucose system in the same engineered bacteria to reduce 2-pyridyl-1-pyrroline to provide products having an optical purity of greater than about 98%. See, e.g., Scheme 1.

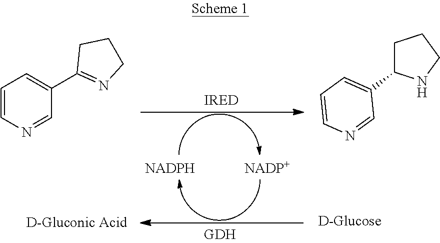 [SMILES: c1cncc(C2=NCCC2)c1 ; c1cncc([C@@H]2CCCN2)c1]

The genetically engineered bacteria expressing a nucleic acid molecule encoding an imine reductase described herein, or expressing a nucleic acid molecule encoding an imine reductase described herein as well as a glucose dehydrogenase, are amplified and cultured in a fermentation culture medium to induce production of target proteins. The recombinant bacteria are then collected. In some embodiments, the bacteria are collected using centrifugation.

The recombinant bacterial cells may be used to form an enzyme solution obtained by breaking down the recombinant bacteria in a buffer solution. In some embodiments, the enzyme solution is a crude solution. Thereafter, 2-pyridyl-1-pyrroline is added to the buffer solution, preferably for reaction with the components of the buffer solution. As used herein, the term “breaking down” refers to subjecting the recombinant bacteria to one or more steps to provide the crude enzyme solution. In some embodiments, the recombinant bacteria is broken down using fermenting, concentrating, resuspending with a buffer, and crushing. In other embodiments, the recombinant bacteria are broken down by fermenting, concentrating, resuspending with a buffer, and crushing by a high-pressure homogenizer or sonicator to obtain the crude enzyme solution. The fermentation culture medium may be water, fermentation broth, or an aqueous medium containing different buffer solutions. In some embodiments, the medium is water. In other embodiments, the medium is a fermentation broth. In further embodiments, the medium is an aqueous medium containing different buffer solutions.

The buffer solution may be a combination of one or more of phosphate, Tris hydrochloride, bicarbonate, and carbonate added to water. In some embodiments, the buffer solution is phosphate in water. In other embodiments, the buffer solution is tris hydrochloride in water. In further embodiments, the buffer solution is carbonate in water.

Preferably, the reaction temperature is kept within a temperature range that enables the imine reductases to express their activity, such as preferably about 20° C. to about 40° C. In some embodiments, the temperature is about 20° C. In other embodiments, the temperature is about 25° C. In still other embodiments, the temperature is about 28° C. In further embodiments, the temperature is about 30° C. In still other embodiments, the temperature is about 35° C. In still other embodiments, the temperature is about 37° C. In yet further embodiments, the temperature is about 40° C.

After the reaction is complete, a supernatant is collected. The pH value may then be adjusted using skill in the art. For example, an inorganic base may be utilized to adjust the pH.

The inorganic base for basification is a combination of one or more of sodium hydroxide, potassium hydroxide, and sodium carbonate. In some embodiments, the inorganic base is sodium hydroxide. In other embodiments, the inorganic base is potassium hydroxide. In further embodiments, the inorganic base is sodium carbonate.

Preferably, the pH is maintained within a range that enables the imine reductases to express their activity, such as about 6 to about 10. In some embodiments, the pH is about 6. In other embodiments, the pH is about 7. In further embodiments, the pH is about 8. In yet other embodiments, the pH is about 9. In still further embodiments, the pH is about 10.

The substrate concentration is not limited. Usually, the substrate concentration is about 10 g/L to about 90 g/L. Considering the reaction effect, the substrate concentration is preferably greater than or equal to 50 g/L. In some embodiments, the concentration is about 10 g/L. In other embodiments, the concentration is about 20 g/L. In further embodiments, the concentration is about 30 g/L. In yet other embodiments, the concentration is about 40 g/L. In still further embodiments, the concentration is about 50 g/L. In other embodiments, the concentration is about 60 g/L. In further embodiments, the concentration is about 70 g/L. In yet other embodiments, the concentration is about 80 g/L. In still further embodiments, the concentration is about 90 g/L. In addition, to improve production efficiency, the substrate may be added in batches to the reaction. The reaction product may also be separated after the reaction is completed, i.e., collected. Alternatively, the product may be continuously removed and used in situ in latter reactions, i.e., without isolating the reaction product.

One or more extractions are then performed using the supernatant to provide organic phases. In some embodiments, the extractions are performed using an organic solvent. The organic solvent for extraction may be one or more of dichloromethane, ethyl acetate, methyl tert-butyl ether. In some embodiments, the organic solvent is dichloromethane. In other embodiments, the organic solvent is ethyl acetate. In further embodiments, the organic solvent is methyl tert-butyl ether.

The organic phases are then combined once or a plurality of times. In some embodiments, there is one organic phase collected from the extraction. In other embodiments, there are more than one organic phases collected from the extraction.

The one or more organic phases are then dried. In some embodiments, the drying is performed using a desiccant. The desiccant may be selected by one skilled in the art. For example, the desiccant may be anhydrous sodium sulfate, anhydrous magnesium sulfate, or the like. In some embodiments, the desiccant is anhydrous sodium sulfate. In other embodiments the desiccant is anhydrous magnesium sulfate.

Once dried, the solution is filtered using skill in the art.

After filtering, the solvent is removed to obtain a target product. In some embodiments, the solvent is removed using rotary-evaporation.

In the present disclosure, the genetically engineered bacteria that produce imine reductases are cultured in a seed culture medium and inoculated into a fermentation culture medium at a specific ratio. After a specific period of time, an inducer such as isopropyl β-D-1-thiogalactopyranoside (IPTG), lactose, or a mixture of both, is added to induce the culture for a specific period of time, and bacteria are collected through centrifugation for high pressure breakage. Transformation is performed for 2 to 24 hours in the following reaction conditions: the buffer solution having a pH value of 6.0 to 10.0, a 2-pyridyl-1-pyrroline substrate having a concentration of 10 g/L to 100 g/L, a reaction temperature of 20° C. to 40° C., and a rotation speed of 200 rpm. After the reaction is completed, centrifugation, basification, extraction, and desolvation are performed to obtain (S)-2-(3-pyridyl)-pyrrolidine, with a yield greater than 80%.

The present disclosure relates to methods for biocatalytic synthesis of (S)-2-(3-pyridyl)-pyrrolidine. More specifically, the present disclosure provides methods for reducing 2-pyridyl-1-pyrroline compounds to (S)-2-(3-pyridyl)-pyrrolidines using imine reductases derived from Myxococcus fulvus and genetically engineered bacteria of the imine reductases. The imine reductases derived from Myxococcus fulvus have high activity, a reaction substrate concentration, a reaction yield. Additionally, optical purity of the product is high, and in the reaction process, operations are simple and energy consumption is low. Therefore, the imine reductases derived from Myxococcus fulvus meet the requirements of green chemistry as defined by the US Environmental Protection Agency and can be used for biotransformation and preparation of (S)-2-(3-pyridyl)-pyrrolidine compounds in industrial production.

### Methods of Using the Imine Reductases for Preparing (S)-Nicotine

The present disclosure provides methods for preparing (S)-nicotine using novel enzyme catalysis technology. By doing so, (S)-nicotine with high yields and high optical purities can be obtained. Further, the (S)-nicotine may be prepared using a one-pot multi-step integrated method, without the need to separately isolate each intermediate. The steps are shorter than other synthesis routes. Advantageously, the methods for preparing (S)-nicotine avoid the need to use hazardous chemicals, e.g., butyl lithium and lithium hexamethyldisilazide, thereby avoiding safety issues, harsh reaction conditions, high toxicity, and the like. In addition, less industrial wastewater, waste gases and residues are produced, production cost is low, and production safety is controllable. The novel enzymes can be efficiently reused, which is beneficial for several reasons.

The methods also advantageously permit the direct preparation of (S)-nicotine, avoiding the need for unnecessary resolution steps. Finally, the methods are short and the total yield of (S)-nicotine is high.

Thus, the present disclosure provides methods for preparing nicotine. The term “nicotine” as used herein refers 3-(1-methyl-2-pyrrolidinyl)pyridine having the following structure:

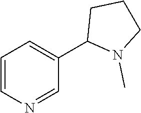

In some embodiments, the nicotine is (S)-nicotine. (S)-nicotine refers (S)-3-(1-methyl-2-pyrrolidinyl)pyridine having the following structure:

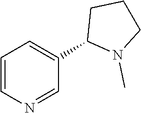

Advantageously, the methods result in (S)-nicotine with high optical purities. In some embodiments, the methods provide (S)-nicotine with an optical purity of at least about 90% ee (“enantiomeric excess”). In other embodiments, the methods provide (S)-nicotine with an optical purity of at least about 90% ee, about 91% ee, about 92% ee, about 93% ee, about 94% ee, about 95% ee, about 96% ee, about 97% ee, about 98% ee, about 99% ee, or about 100% ee. Thus, the methods provide products have less than about 10% ee of (R)-nicotine. In other embodiments, the methods provide (R)-nicotine with an optical purity of less than about 10% ee, about 9% ee, about 8% ee, about 7% ee, about 6% ee, about 5% ee, about 4% ee, about 3% ee, about 2% ee, about 1% ee, or about 0 ee.

As described in detail herein, (S)-nicotine is prepared as shown in Scheme 2, wherein M is an alkali metal and n is 0 or greater. In some embodiments, n is 0-10. In other embodiments, n is 0, 1, 2, 3, 4, 5, 6, 7, 8, 9, or 10. In further embodiments, n is 0.

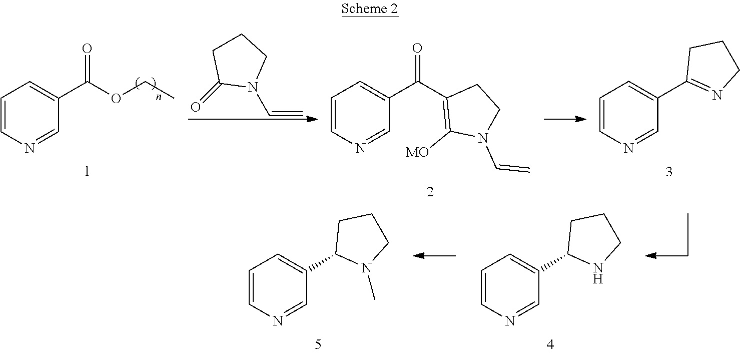

The series of steps of Scheme 2 may be performed individually with isolating each of intermediate compounds 2, 3, and 4. Alternatively, the series of steps in Scheme 2 are performed in a one-pot, i.e., without isolating each intermediate compound. In some embodiments, compound 2 is isolated. In further embodiments, compound 3 is isolated. In further embodiments, compound 4 is isolated. In other embodiments, none of compounds 2, 3, and 4 are isolated.

In this scheme, nicotinic acid ester (1) and vinyl pyrrolidone are condensed to obtain a salt of 3-nicotinoyl-1-vinyl-4,5-dihydro-1H-pyrrol-2-olate (2). The 3-nicotinoyl-1-vinyl-4,5-dihydro-1H-pyrrol-2-olate salt is then converted to myosmine (3). The myosmine (3) is subjected to catalytic reduction under a biological enzyme system to obtain nornicotine (4). The pyrrole ring of nornicotine (4) is then methylated to obtain (S)-nicotine (S).

The methods include condensing a nicotinic acid ester and vinyl pyrrolidone to provide a salt of 3-nicotinoyl-1-vinyl-4,5-dihydro-1H-pyrrol-2-olate. In some aspects, the salt of 3-nicotinoyl-1-vinyl-4,5-dihydro-1H-pyrrol-2-olate is a sodium, potassium, or lithium salt. In other aspects, the salt of 3-nicotinoyl-1-vinyl-4,5-dihydro-1H-pyrrol-2-olate is potassium 3-nicotinoyl-1-vinyl-4,5-dihydro-1H-pyrrol-2-olate. In further aspects, the salt is lithium 3-nicotinoyl-1-vinyl-4,5-dihydro-1H-pyrrol-2-olate.

Examples of nicotinic acid esters include, without limitation, methyl nicotinate, ethyl nicotinate, or tert-butyl nicotinate. In some embodiments, the nicotinic acid ester is methyl nicotinate. In other embodiments, the nicotinic acid ester is ethyl nicotinate. In further embodiments, the nicotinic acid ester is tert-butyl nicotinate. The condensation is performed in presence of a strong base. Examples of strong bases include, without limitation, sodium hydroxide, potassium hydroxide, sodium hydride, sodium tert-butoxide, potassium tert-butoxide, or lithium tert-butoxide. In some embodiments, the strong base is sodium hydroxide. In other embodiments, the strong base is potassium hydroxide. In further embodiments, the strong base is sodium hydride. In yet other embodiment, the strong base is sodium tert-butoxide. In still further embodiments, the strong base is potassium tert-butoxide. In other embodiments, the strong base is lithium tert-butoxide. Preferably, the strong base is potassium tert-butoxide.

In certain embodiments, the vinylpyrrolidone, and potassium tert-butoxide are added to an organic solvent, such as n-hexane or toluene. In some embodiments, the solvent is n-hexane. In other embodiments, the solvent is toluene. The reaction may performed at elevated temperatures, e.g., the reflux temperature of the solvent, for a time sufficient to form the salt of 3-nicotinoyl-1-vinyl-4,5-dihydro-1H-pyrrol-2-olate. For example, the temperature is about 50° C. to 70° C. In some embodiments, the temperature is about 50° C., 55° C., 60° C., 65° C., or about 70° C. In other embodiments, the temperature is about 50° C. to about 70° C., about 50° C. to about 65° C., about 50° C. to about 60° C., about 50° C. to about 55° C., about 55° C. to about 70° C., about 55° C. to about 65° C., about 55° C. to about 60° C., about 60° C. to about 70° C., about 60° C. to about 65° C., or about 65° C. to about 70° C. In further embodiments, the temperature is about 55° C. to about 65° C.

In some embodiments, the reaction is performed for at least about 1 hour. In other embodiments, the reaction is performed for about 1 to about 6 hours. In further embodiments, the reaction is performed for about 1 hour, about 2 hours, about 3 hours, about 4 hours, about 5 hours, or about 6 hours.

The term “reflux” as used herein refers to the reflux temperature of the solvent or mixture of solvents. Such information is understood to those skilled in the art and depends on the solvent or mixture thereof utilized.

Thereafter, routine steps known to those skilled in the art including, without limitation, extraction and concentration may be performed to isolate the 3-nicotinoyl-1-vinyl-4,5-dihydro-1H-pyrrol-2-olate salt. Alternatively, 3-nicotinoyl-1-vinyl-4,5-dihydro-1H-pyrrol-2-olate salt myosmine is not isolated and carried on to the next step.

The 3-nicotinoyl-1-vinyl-4,5-dihydro-1H-pyrrol-2-olate salt is then converted to myosmine. The inventors hypothesize that conversion of the 3-nicotinoyl-1-vinyl-4,5-dihydro-1H-pyrrol-2-olate salt to myosmine proceeds as shown in Scheme 3. In Scheme 3, the 3-nicotinoyl-1-vinyl-4,5-dihydro-1H-pyrrol-2-olate salt undergoes a ring opening (a→b), decarboxylation (b→c), cyclization (c→d), and elimination (d→e).

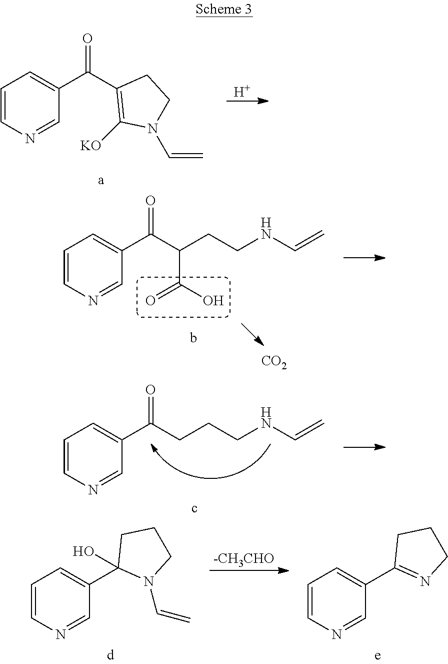

In some embodiments, myosmine is formed by combining the 3-nicotinoyl-1-vinyl-4,5-dihydro-1H-pyrrol-2-olate salt with an acid, such as a dilute acid. Examples of the dilute acid include, without limitation, perchloric acid, sulfuric acid, hydrochloric acid, hydrobromic acid, phosphoric acid, trifluoromethanesulfonic acid, chlorosulfonic acid, sulfamic acid, trifluoroacetic acid, trichloroacetic acid, benzenesulfonic acid, and picric acid, or mixtures thereof. In some aspects, the dilute acid is hydrochloric acid. In other aspects, the dilute acid is perchloric acid. In further aspects, the dilute acid is sulfuric acid. In yet other aspects, the dilute acid is hydrobromic acid. In still further aspects, the dilute acid is phosphoric acid. In other aspects, the dilute acid is trifluoromethanesulfonic acid. In further aspects, the dilute acid is chlorosulfonic acid. In still other aspects, the dilute acid is sulfamic acid. In still other aspects, the dilute acid is trifluoroacetic acid. In yet further aspects, the dilute acid is trichloroacetic acid. In other aspects, the dilute acid is benzenesulfonic acid. In further aspects, the dilute acid is picric acid. In some embodiments, the concentration of the dilute acid is about 3M to about 6M. In other embodiments, the concentration of the dilute acid is about 3, about 4, about 5, or about 6. The reaction is performed for a time sufficient to complete the ring opening. In some embodiments, the reaction is performed for at least about 1 hour. In other embodiments, the reaction is performed for at least about 4 hours, about 8 hours, about 12 hours, about 16 hours, about 20 hours, about 21 hours, about 24 hours, about 28 hours, or about 30 hours.

The reaction may be heated to an elevated temperature, such as reflux. In some embodiments, the reaction is performed at a temperature of about 90° C. to about 110° C. In other embodiments, the reaction is performed at a temperature of about 90° C., about 95° C., about 100° C., about 105° C., or about 110° C. In further embodiments, the reaction is performed at a temperature of about 90° C. to about 105° C., about 90° C. to about 100° C., about 90° C. to about 95° C., about 95° C. to about 110° C., about 95° C. to about 105° C., about 95° C. to about 100° C., about 100° C. to about 110° C., about 100° C. to about 105° C., or about 105° C. to about 110° C. However, one skilled in the art would be able to select a suitable temperature based on the reagents selected.

The myosmine solution is optionally adjusted to a basic pH. In some embodiments, the pH of the myosmine solution is adjusted to a pH of about 10 to about 12. In other embodiments, the pH of the myosmine solution is adjusted to a pH of about 10, 11, or 12. In further embodiments, the pH of the myosmine solution is adjusted to a pH of about 10 to about 11 or about 11 or about 12. The dilute acid solution may be cooled, such as to about room temperature. The solution is then treated with a base that adjusts the pH of the dilute acid solution to about 9 to about 14. In some embodiments, the pH is adjusted to about 9. In other embodiments, the pH is adjusted to about 10. In further embodiments, the pH is adjusted to about 11. In yet other embodiments, the pH is adjusted to about 12. In still further embodiments, the pH is adjusted to about 13. In other embodiments, the pH is adjusted to about 14. Thereafter, routine steps known to those skilled in the art including, without limitation, extraction and concentration may be performed to isolate the myosmine. Alternatively, myosmine is not isolated and carried on to the next step.

Myosmine is then subjected to a catalytic reduction using biological enzyme system to obtain the nornicotine. The term “nornicotine” as used herein refers to 3-[2-pyrrolidinyl]pyridine having the following structure:

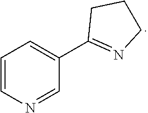 [SMILES: c1cncc(C2=NCCC2)c1]

Nornicotine also includes (S)-nornicotine which refers to 3-[(2S)-2-pyrrolidinyl]pyridine having the following structure:

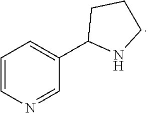 [SMILES: c1cncc(C2CCCN2)c1]

The catalytic reduction may be performed at temperatures of about 20° C. to about 40° C. In some embodiment, the temperature is about 20° C., 25° C., 30° C., 35° C., or 40° C. In other embodiments, the temperature is about 20° C. to about 40° C., about 20° C. to about 37° C., about 20° C. to about 35, about 20° C. to about 30° C., about 20° C. to about 28° C., about 25° C. to about 35° C., about 25° C. to about 30° C., about 30° C. to about 40° C., about 30° C. to about 37° C., or about 35° C. to about 40° C. In further embodiments, the temperature is about 22 to about 37° C. Reaction times for the catalytic reduction may be determined by those skilled in the art. In some embodiments, the catalytic reduction is performed for about 4 hours to about 24 hours, preferably about 8 hours to about 16 hours. In other embodiments, the catalytic reduction is performed for about 8 hours, about 10 hours, about 12 hours, about 14 hours, or about 16 hours.

Thereafter, the pyrrole ring of the nornicotine intermediate is subjected to a methylation to obtain nicotine. The term “methylation” as used herein refers to adding a methyl substituent to a position on nornicotine, such as the nitrogen atom of the pyrrolidine ring. In some embodiments, the methylation is performed using formaldehyde and formic acid, i.e., the Eschweiler-Clarke reaction reagent. One of skill in the art would understand how to perform such a reaction. In some embodiments, an excess of formaldehyde, excess of formic acid, or excess of both formaldehyde and formic acid are utilized. The reaction may be performed at elevated temperatures, such as 50° C. to about 70° C. In some embodiments, the elevated temperature is about 50° C., about 55° C., about 60° C., about 65° C., or about 70° C. In other embodiments, the elevated temperature is about 50° C. to about 70° C., about 50° C. to about 65° C., about 50° C. to about 60° C., about 50° C. to about 55° C., about 55° C. to about 70° C., about 55° C. to about 65° C., about 55° C. to about 60° C., about 60° C. to about 70° C., about 60° C. to about 65° C., about 65° C. to about 70° C. In further embodiments, the elevated temperature is about 60° C. to about 65° C.

(S)-Nicotine is then isolated using skill in the art. The pH of the solution may adjusted to about 9 to about 14. In some embodiments, the pH is adjusted to about 9. In other embodiments, the pH is adjusted to about 10. In further embodiments, the pH is adjusted to about 11. In yet other embodiments, the pH is adjusted to about 12. In still further embodiments, the pH is adjusted to about 13. In other embodiments, the pH is adjusted to about 14. Thereafter, the (S)-nicotine is obtained through standard techniques.

The nicotine may then be purified using techniques known to those skilled in the art. In some embodiments, the purification comprises distillation, optionally under reduced pressure. The distillation is performed at elevated temperatures such as about 110° C. to about 125° C. In some embodiments, the distillation is performed at about 110° C., about 115° C., about 120° C., or about 125° C. In further embodiments, the distillation is performed at about 110° C. to about 125° C., about 110° C. to about 120° C., about 110° C. to about 115° C., about 115° C. to about 125° C., about 115° C. to about 120° C., or about 120° C. to about 125° C.

The methods thereby provide (S)-nicotine in high optical purities, for example, of about at least about 95%. In some embodiments, the optical purity is at least about 96%, at least about 97%, at least about 98%, at least about 99%, or at least about 99.5%. In other embodiments, the optical purity is 100%, i.e., the single chiral (S)-nicotine. Advantageously, the methods provide (S)-nicotine in optical purities that are consistent with that of natural extraction products. In addition to high optical purities, the methods provide high yields of (S)-nicotine. In some embodiments, the methods result in yields of (S)-nicotine of about 80% or greater. In certain embodiments, the (S)-nicotine yield is at least about 85%, at least about 90%, at least about 95%, at least about 98%, at least about 99%.

### Aspects I

Aspect 1. A nucleic acid molecule encoding an imine reductase of Myxococcus fulvus according to SEQ ID NO: 2 and comprising a nucleotide sequence codon optimized for heterologous expression.

Aspect 2. The nucleic acid molecule of Aspect 1, wherein the heterologous expression is expression in a bacterial host cell.

Aspect 3. The nucleic acid molecule of Aspect 1 or 2, wherein the heterologous expression is expression in Escherichia coli host cell selected from the group consisting of expression strains BL21, ROSETTA™, ORIGAMI™, and TUNER™ strains.

Aspect 4. The nucleic acid molecule of any one of Aspects 1-3 comprising a nucleotide sequence according to SEQ ID NO: 1.

Aspect 5. The nucleic acid molecule of any one of Aspects 1˜4 in an expression vector.

Aspect 6. The nucleic acid molecule of Aspect 5, wherein the expression vector is selected from the group consisting of a pET series plasmid, a pTXB series plasmid, a pGEX series plasmid, a pETduet series plasmid, a pTYB series plasmid, a pRSF series plasmid, a pET-28a(+) plasmid, and a pRSFDuet1 plasmid.

Aspect 7. The nucleic acid molecule of Aspect 5 or 6, wherein the expression vector is pRSFDuet1.

Aspect 8. The nucleic acid molecule of any one of Aspects 5-7, wherein the expression vector comprises a nucleic acid molecule encoding glucose dehydrogenase.

Aspect 9. A nucleic acid molecule encoding a mutant imine reductase of Myxococcus fulvus comprising an amino acid sequence according to any one of SEQ ID NOs: 3-19.

Aspect 10. The nucleic acid molecule of Aspect 9 comprising a nucleotide sequence according to SEQ ID NOs: 1 and mutated to encode an amino acid sequence according to any one of SEQ ID NOs: 3-19.

Aspect 11. The nucleic acid molecule of Aspect 9 or 10 in an expression vector.

Aspect 12. The nucleic acid molecule of Aspect 11, wherein the expression vector is selected from the group consisting of a pET series plasmid, a pTXB series plasmid, a pGEX series plasmid, a pETduet series plasmid, a pTYB series plasmid, a pET-28a(+) plasmid, and a pRSFDuet1 plasmid.

Aspect 13. The nucleic acid molecule of Aspect 11 or 12, wherein the expression vector is pRSFDuet1.

Aspect 14. The nucleic acid molecule of any one of Aspects 11-13, wherein the expression vector comprises a nucleic acid molecule encoding glucose dehydrogenase.

Aspect 15. A bacterial host cell comprising the nucleic acid molecule of any one of Aspects 1-14.

Aspect 16. A bacterial host cell comprising the nucleic acid molecule of any one of Aspects 1-14 and an imine reductase having an amino acid sequence of any one of SEQ ID NOs: 2-19.

Aspect 17. A crude enzyme solution comprising an imine reductase having an amino acid sequence of any one of SEQ ID NOs: 2-19.

Aspect 18. A method of producing an imine reductase of Myxococcus fulvus according to SEQ ID NO: 2, or an imine reductase having an amino acid sequence of any one of SEQ ID NOs: 3-19, in bacterial host cells, the method comprising

a) culturing bacterial host cells comprising the nucleic acid molecule of any one of Aspects 1-14 in a fermentation culture medium at a first temperature;

b) culturing the bacterial host cells in the fermentation culture medium at a second temperature; and

c) collecting the bacterial host cells.

Aspect 19. The method of Aspect 18, wherein the fermentation medium comprises an induction compound selected from the group consisting of isopropyl β-d-1-thiogalactopyranoside (IPTG) and lactose.

Aspect 20. The method of Aspect 18 or 19, wherein culturing in the fermentation culture medium at the first temperature comprises culturing at between about 30° C. and 40° C. with rotation speed of between about 150 rotations per minute (rpm) and 250 rpm.

Aspect 21. The method of any one of Aspects 18-20, wherein culturing in the fermentation culture medium at the first temperature comprises culturing until optical density at 600 nm (OD600) of the fermentation medium is about or greater than 2.0.

Aspect 22. The method of any one of Aspects 18-21, wherein culturing in the fermentation culture medium at the second temperature comprises culturing at between about 20° C. and 30° C. with rotation speed of between about 150 rotations per minute (rpm) and 250 rpm.

Aspect 23. The method of any one of Aspects 18-22, wherein culturing in the fermentation culture medium at the second temperature comprises culturing for a period between about 1 hour and about 24 hours.

Aspect 24. The method of any one of Aspects 18-23, wherein collecting is by centrifugation.

Aspect 25. A method of biocatalytic synthesis of a chiral (s)-2-(3-pyridyl)-pyrrolidine comprising combining and reacting the bacterial host cell of Aspect 15 or 16, or the crude enzyme solution of Aspect 17, with 2-pyridyl-1-pyrroline in a reaction system.

Aspect 26. The method of Aspects 25, wherein combining and reacting comprises adding the bacterial host cell of Aspect 15 or 16, or the crude enzyme solution of Aspect 17, to a buffer solution, and adding 2-pyridyl-1-pyrroline to the buffer solution in the reaction system.

Aspect 27. The method of Aspect 25 or 26 comprising collecting a supernatant from the reaction system.

Aspect 28. The method of any one of Aspects 25-27 comprising adjusting the pH value to above 7.0.

Aspect 29. The method of any one of Aspects 25-28 comprising extracting organic phases using an organic solvent.

Aspect 30. The method of any one of Aspects 25-29 comprising drying organic phases to obtain a chiral (s)-2-(3-pyridyl)-pyrrolidine.

Aspect 31. The method of any one of Aspects 25-30, wherein the 2-pyridyl-1-pyrroline is at a concentration of 10 g/L to 100 g/L in the step of combining.

Aspect 32. The method of any one of Aspects 25-31, wherein the reacting occurs at a reaction temperature between about 20° C. and about 40° C. and a rotation speed of about 200 rpm.

Aspect 33. The method of any one of Aspects 25-32, wherein the reacting occurs for a period of time between about 1 hour and about 24 hours.

Aspect 34. The method of any one of Aspects 25-33, wherein the reacting occurs at pH at or greater than about 6.0.

Aspect 35. The method of any one of Aspects 26-34, wherein the buffer solution has a pH value between about 6.0 and about 10.0.

Aspect 36. The method of any one of Aspects 26-35, wherein the buffer solution comprises a buffer selected from the group consisting of a phosphate buffer, Tris hydrochloride, bicarbonate buffer, and carbonate added to water.

Aspect 37. The method of any one of Aspects 25-36, wherein the reaction system comprises nicotinamide adenine dinucleotide phosphate (NADP+), glucose, and glucose dehydrogenase.

Aspect 38. The method of any one of Aspects 25-37 having a yield greater than 80%.

### Aspects II

Aspect 1-II. A preparation method of nicotine with a high optical purity, comprising:
(a) condensing nicotinic acid ester and vinyl pyrrolidone in presence of a strong alkaline substance to obtain potassium 3-nicotinoyl-1-vinyl-4,5-dihydro-1H-pyrrol-2-olate;
(b) subjecting the potassium 3-nicotinoyl-1-vinyl-4,5-dihydro-1H-pyrrol-2-olate to reflux stirring in an acidic compound with a dilute concentration, followed through a one-pot reaction, to obtain Myosmine;
(c) subjecting the Myosmine to catalytic reduction under a biological enzyme system to obtain an intermediate with a high optical purity, nornicotine; and
(d) subjecting a pyrrole ring of the nornicotine to an aminomethylation reaction to obtain an end product, (S)-nicotine.

Aspect 2-II. The preparation method according to Aspect 1-II, wherein the nicotinic acid ester is methyl nicotinate, ethyl nicotinate, or tert-butyl nicotinate.

Aspect 3-II. The preparation method according to Aspect 2-II, wherein the strong alkaline substance is sodium hydroxide, potassium hydroxide, sodium hydride, sodium tert-butoxide, potassium tert-butoxide, or lithium tert-butoxide.

Aspect 4-II. The preparation method according to Aspect 2-II, wherein the acidic compound is one or a mixture of two or more of perchloric acid, sulfuric acid, hydrochloric acid, hydrobromic acid, phosphoric acid, trifluoromethanesulfonic acid, chlorosulfonic acid, sulfamic acid, trifluoroacetic acid, trichloroacetic acid, benzenesulfonic acid, and picric acid.

Aspect 5-II. The preparation method according to Aspect 4-II, wherein a concentration of the hydrochloric acid is 3 M to 6 M.

Aspect 6-II. The preparation method according to Aspect 4-II, wherein the one-pot reaction comprises a ring opening reaction, a decarboxylation reaction, and a cyclization reaction.

Aspect 7-II. The preparation method according to Aspect 6-II, wherein after the Myosmine generated during the reflux stirring is cooled, an alkali is added to adjust pH to 9 to 14, and the Myosmine is obtained through extraction and concentration.

Aspect 8-II. The preparation method according to Aspect 7-II, wherein the biological enzyme catalysis system involves biological enzyme cyclic catalysis, and the Myosmine is subjected to reduction by the biological enzyme cyclic catalysis to obtain the intermediate with a high optical purity, nornicotine.

Aspect 9-II. The preparation method according to Aspect 8-II, wherein the biological enzyme system comprises glucose dehydrogenase and imine reductase.

Aspect 10-II. The preparation method according to Aspect 9-II, wherein an optical purity of the prepared (S)-nicotine reaches 99% or above.

Aspect 11-II. The preparation method according to Aspect 10-II, wherein a reaction temperature of the biological enzyme catalysis system is 22° C. to 37° C.

Aspect 12-II. The preparation method according to Aspect 11-II, wherein a reaction time of the biological enzyme catalysis system is 8 to 16 hours.

Aspect 13-II. The preparation method according to Aspect 11-II, wherein after the subjecting a pyrrole ring of the nornicotine to an aminomethylation reaction, pH is first adjusted to 9 to 14, and the end product, (S)-nicotine, is obtained through extraction with an extraction agent, concentration, and distillation.

Aspect 14-II. The preparation method according to Aspect 7-II or Aspect 13-II, wherein the extraction agent used in the extraction is one or a mixture of two or more of n-hexane, ethyl acetate, dichloromethane, chloroform, and butyl acetate.

### Aspects III

Aspect III-1. A nucleic acid molecule encoding an imine reductase of Myxococcus fulvus according to SEQ ID NO: 2 and comprising a nucleotide sequence codon optimized for heterologous expression.

Aspect III-2. The nucleic acid molecule of Aspect III-1, wherein the heterologous expression is expression in a bacterial host cell.

Aspect III-3. The nucleic acid molecule of Aspect III-1 or 2, wherein the heterologous expression is expression in Escherichia coli host cell selected from the group consisting of expression strains BL21, ROSETTA™, ORIGAMI™, and TUNER™ strains.

Aspect III-4. The nucleic acid molecule of any one of Aspects III-1-3 comprising a nucleotide sequence according to SEQ ID NO: 1.

Aspect III-5. The nucleic acid molecule of any one of Aspects III-1-4 in an expression vector.

Aspect III-6. The nucleic acid molecule of Aspect III-5, wherein the expression vector is selected from the group consisting of a pET series plasmid, a pTXB series plasmid, a pGEX series plasmid, a pETduet series plasmid, a pTYB series plasmid, a pET-28a(+) plasmid, and a pRSFDuet1 plasmid.

Aspect III-7. The nucleic acid molecule of Aspect III-5 or 6, wherein the expression vector is pRSFDuet1.

Aspect III-8. The nucleic acid molecule of any one of Aspects III-5-7, wherein the expression vector comprises a nucleic acid molecule encoding glucose dehydrogenase.

Aspect III-9. A nucleic acid molecule encoding a mutant imine reductase of Myxococcus fulvus comprising an amino acid sequence according to any one of SEQ ID NOs: 3-19.

Aspect III-10. The nucleic acid molecule of Aspect III-9 comprising a nucleotide sequence according to SEQ ID NOs: 1 and mutated to encode an amino acid sequence according to any one of SEQ ID NOs: 3-19.

Aspect III-11. The nucleic acid molecule of Aspect III-9 or 10 in an expression vector.

Aspect III-12. The nucleic acid molecule of Aspect III-11, wherein the expression vector is selected from the group consisting of a pET series plasmid, a pTXB series plasmid, a pGEX series plasmid, a pETduet series plasmid, a pTYB series plasmid, a pRSF series plasmid, a pET-28a(+) plasmid, and a pRSFDuet1 plasmid.

Aspect III-13. The nucleic acid molecule of Aspect III-11 or 12, wherein the expression vector is pRSFDuet1.

Aspect III-14. The nucleic acid molecule of any one of Aspects III-11-13, wherein the expression vector comprises a nucleic acid molecule encoding glucose dehydrogenase.

Aspect III-15. A bacterial host cell comprising the nucleic acid molecule of any one of Aspects III-1-14.

Aspect III-16. A bacterial host cell comprising the nucleic acid molecule of any one of Aspects III-1-14 and an imine reductase having an amino acid sequence of any one of SEQ ID NOs: 2-19.

Aspect III-17. A crude enzyme solution comprising an imine reductase having an amino acid sequence of any one of SEQ ID NOs: 2-19.

Aspect III-18. A method of producing an imine reductase of Myxococcus fulvus according to SEQ ID NO: 2, or an imine reductase having an amino acid sequence of any one of SEQ ID NOs: 3-19, in bacterial host cells, the method comprising

a) culturing bacterial host cells comprising the nucleic acid molecule of any one of Aspects III-1-14 in a fermentation culture medium at a first temperature;

b) culturing the bacterial host cells in the fermentation culture medium at a second temperature; and

c) collecting the bacterial host cells.

Aspect III-19. The method of Aspect III-18, wherein the fermentation medium comprises an induction compound selected from the group consisting of isopropyl β-d-1-thiogalactopyranoside (IPTG) and lactose.

Aspect III-20. The method of Aspect III-18 or 19, wherein culturing in the fermentation culture medium at the first temperature comprises culturing at between about 30° C. and 40° C. with rotation speed of between about 150 rotations per minute (rpm) and 250 rpm.

Aspect III-21. The method of any one of Aspects III-18-20, wherein culturing in the fermentation culture medium at the first temperature comprises culturing until optical density at 600 nm (OD600) of the fermentation medium is about or greater than 2.0.

Aspect III-22. The method of any one of Aspects III-18-21, wherein culturing in the fermentation culture medium at the second temperature comprises culturing at between about 20° C. and 30° C. with rotation speed of between about 150 rotations per minute (rpm) and 250 rpm.

Aspect III-23. The method of any one of Aspects III-18-22, wherein culturing in the fermentation culture medium at the second temperature comprises culturing for a period between about 1 hour and about 24 hours.

Aspect III-24. The method of any one of Aspects III-18-23, wherein collecting is by centrifugation.

Aspect III-25. A method of preparing 2-(3-pyridyl)-pyrrolidine, comprising combining the bacterial host cell of Aspect III-15 or 16, or the crude enzyme solution of Aspect III-17, with 2-pyridyl-1-pyrroline.

Aspect III-26. The method of Aspect III-25, wherein the 2-(3-pyridyl)-pyrrolidine is (S)-2-(3-pyridyl)-pyrrolidine.

Aspect III-27. The method of Aspect III-25, comprising combining the bacterial host cell of Aspect III-15 or 16, or the crude enzyme solution of Aspect III-17, with a buffer solution, and adding 2-pyridyl-1-pyrroline to the buffer solution.

Aspect III-28. The method of any one of Aspects III-25-27 comprising adjusting the pH value to above 7.0.

Aspect III-29. The method of any one of Aspects III-25-28, wherein the method occurs at a reaction temperature between about 20° C. and about 40° C. and a rotation speed of about 200 rpm.

Aspect III-30. The method of any one of Aspects III-25-29, wherein the method occurs at pH at or greater than about 6.0.

Aspect III-31. The method of any one of Aspects III-26-30, wherein the buffer solution has a pH value between about 6.0 and about 10.0.

Aspect III-32. The method of any one of Aspects III-26-31, wherein the buffer solution comprises a buffer selected from the group consisting of a phosphate buffer, Tris hydrochloride, bicarbonate buffer, and carbonate added to water.

Aspect III-33. The method of any one of Aspects III-25-32, wherein the reaction system comprises nicotinamide adenine dinucleotide phosphate (NADP+), glucose, and glucose dehydrogenase.

Aspect III-34. A method for preparing nornicotine, comprising contacting myosmine with the bacterial host cell of Aspect III-15 or 16, or the crude enzyme solution of Aspect III-17.

Aspect III-35. The method of Aspect III-34, further comprising glucose dehydrogenase.

Aspect III-36. The method of Aspect III-34 or 35, further comprising adding glucose and NAD(P)-2Na.

Aspect III-37. The method of any one of Aspects III-34-36, wherein the nornicotine is (S)-nornicotine.

Aspect III-38. The method of any one of Aspects III-34-37, that is performed at a temperature of about 20° C. to about 40° C., or about 22° C. to about 37° C.

Aspect III-39. The method of any one of Aspects III-34-38, further comprising methylating the nornicotine to provide nicotine.

Aspect III-40. The method of Aspect III-39, wherein the methylating is performed using formaldehyde and formic acid.

Aspect III-41. The method of Aspect III-39 or 40, that is performed at a temperature of about 50° C. to about 70° C., preferably about 60° C. to about 65° C.

Aspect III-42. The method of any one of Aspects III-39-41, wherein the nicotine is purified.

Aspect III-43. The method of Aspect III-42, wherein the purification comprises distilling, optionally under reduced pressure.

Aspect III-44. The method of Aspect III-43, wherein the distilling is performed at a temperature of about 110° C. to about 125° C.

Aspect III-45. The method of any one of Aspects III-39-44, wherein the nicotine is isolated.

Aspect III-46. The method of any one of Aspects III-39-45, wherein the nicotine is (S)-nicotine.

Aspect III-47. The method of Aspect III-46, wherein the (S)-nicotine has an optical purity of at least about 95% ee, preferably at least about 99% ee.

Aspect III-48. The method of any one of Aspects III-34-47, further comprising contacting a 3-nicotinoyl-1-vinyl-4,5-dihydro-1H-pyrrol-2-olate salt with a dilute acid to provide the myosmine.

Aspect III-49. The method of Aspect III-48, wherein the dilute acid is perchloric acid, sulfuric acid, hydrochloric acid, hydrobromic acid, phosphoric acid, trifluoromethanesulfonic acid, chlorosulfonic acid, sulfamic acid, trifluoroacetic acid, trichloroacetic acid, benzenesulfonic acid, and picric acid, or mixtures thereof, preferably hydrochloric acid.

Aspect III-50. The method of Aspect III-48 or 49, wherein the dilute acid has a concentration of about 3M to about 6M.

Aspect III-51. The method of any one of Aspects III-48-50, comprising heating to a temperature of about 90° C. to about 110° C., preferably about 95° C. to about 105° C.

Aspect III-52. The method of any one of Aspects III-48-51, wherein the 3-nicotinoyl-1-vinyl-4,5-dihydro-1H-pyrrol-2-olate salt is a sodium, potassium, or lithium salt.

Aspect III-53. The method of any one of Aspects III-48-52, wherein the 3-nicotinoyl-1-vinyl-4,5-dihydro-1H-pyrrol-2-olate salt is potassium 3-nicotinoyl-1-vinyl-4,5-dihydro-1H-pyrrol-2-olate or lithium 3-nicotinoyl-1-vinyl-4,5-dihydro-1H-pyrrol-2-olate.

Aspect III-54. The method of any one of Aspects III-48-53, wherein the pH of the myosmine is adjusted to about 10 to about 12.

Aspect III-55. The method of any one of Aspects III-48-54, further comprising combining a nicotinic acid ester and N-vinyl-2-pyrrolidone, and an alkali metal base to provide the 3-nicotinoyl-1-vinyl-4,5-dihydro-1H-pyrrol-2-olate salt.

Aspect III-56. The method of Aspect III-55, wherein the nicotinic acid ester is methyl nicotinate, ethyl nicotinate, or tert-butyl nicotinate, preferably methyl nicotinate.

Aspect III-57. The method of Aspect III-55, wherein the alkali metal base is sodium hydroxide, potassium hydroxide, sodium hydride, sodium tert-butoxide, potassium tert-butoxide, or lithium tert-butoxide, or combinations thereof.

Aspect III-58. The method of any one of Aspects III-55-57, comprising heating to a temperature of about 50 to 70° C., preferably about 55 to about 65° C.

Aspect III-59. A method for preparing nicotine, comprising:

(i) combining a nicotinic acid ester and N-vinyl-2-pyrrolidone, and an alkali metal base to provide 3-nicotinoyl-1-vinyl-4,5-dihydro-1H-pyrrol-2-olate salt;

(ii) contacting the 3-nicotinoyl-1-vinyl-4,5-dihydro-1H-pyrrol-2-olate salt with a dilute acid to provide myosmine;

(iii) contacting the myosmine with the bacterial host cell of Aspect III-15 or 16, or the crude enzyme solution of Aspect III-17 to provide nornicotine; and

(iv) methylating the nornicotine to provide nicotine.

Aspect III-60. The method of Aspect III-59, wherein the nornicotine is (S)-nornicotine.

Aspect III-61. Nicotine prepared according to any one of the methods of Aspects III-34-60.

Aspect III-62. (S)-Nicotine prepared according to any one of the methods of Aspects III-34-60.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### EXAMPLES

### Example 1: Obtaining High-Expression Genetically Engineered Bacteria

Whole gene synthesis was completed by General Biosystems (Anhui) Co., Ltd.

Codon optimization was performed on imine reductases MsIR1 (WP_074958336.1) of the bacterium (Myxococcus fulvus), so that the gene could be expressed in an Escherichia coli expression host. Nde I and EcoR I restriction enzyme cutting sites were added at both ends of the gene to allow for insertion into the pET-28a(+) vector to obtain genetically engineered bacteria M1.

The prepared recombinant vector was transformed into Escherichia coli BL21, ROSETTA™, or ORIGAMI™ using conventional methods to yield genetically engineered bacteria in which the recombinant imine reductases were present in a soluble form, and successfully constructed genetically engineered bacteria were identified. The target protein of the recombinant bacteria in which Escherichia coli BL21 was the host bacterium was better expressed. Engineered bacteria with a target protein expression level of not less than 20% were used as an engineered bacterial strain for production and were stored in glycerol or freeze-dried milk.

### Example 2: Genetically Engineered Bacteria Culture and Crude Enzyme Solution Preparation

A single plated colony of the recombinant bacteria of Example 1 was inoculated into a 5 ml fermentation culture medium containing corresponding antibiotics for about 15 h of culture to obtain a seed solution. The seed solution was inoculated into a 600 ml fermentation culture medium with an inoculation amount of 1% and then cultured on a 200-rpm shaker at 37° C. to OD600=0.6 to 0.8. IPTG with a final concentration of 0.1 mM was added for induction for more than 10 h. The culture solution was centrifuged at 8000 rpm to collect bacteria, and high pressure breakage was performed to obtain the crude enzyme solution of the imine reductases.

### Example 3: Synthesis of (S)-2-(3-pyridyl)-pyrrolidine Through Imine Reductase Whole-Cell Catalysis

A 10 ml phosphate buffer solution (pH 7.5), 30 mg/ml recombinant bacteria from Example 1, 2 eq glucose, 0.2 mg/ml NADP⁺, and 10 mg glucose dehydrogenase (GDH) powder were reacted at 28° C. with a substrate concentration of 50 mg/ml. The TLC dot plate was used to determine the reaction progress. After 12 hours, a saturated sodium hydroxide solution was added to adjust the pH value to above 10, and the denatured protein was removed through centrifugation. The supernatant was extracted with dichloromethane, dried, and spin-dried to collect the product, and then HPLC detection was performed. See, Table 1.

TABLE 1

| No. | pH | Conversion rate | ee (%) |
|---|---|---|---|
| 1 | 6.0 | 26.1 | 99.7 |
| 2 | 6.5 | 48.9 | 99.8 |
| 3 | 7.0 | 84.6 | 99.7 |
| 4 | 7.5 | 98.7 | 99.8 |
| 5 | 8.0 | 89.1 | 99.8 |
| 6 | 8.5 | 80.3 | 99.7 |
| 7 | 9.0 | 72.6 | 99.7 |
| 8 | 9.5 | 55.8 | 99.7 |

Table 1 shows that the conversion rate was higher when the pH of the buffer solution ranged from 7.0 to 9.0. Further, the conversion rate was significantly higher when the pH ranged from 7.5 to 8.0.

### Example 4: Synthesis of (S)-2-(3-Pyridyl)-Pyrrolidine Through Imine Reductase Whole-Cell Catalysis

A 10 ml phosphate buffer solution (pH 7.5), 60 mg/ml recombinant bacteria from Example 1, 2 eq glucose, 0 mg/ml to 0.8 mg/ml NADP⁺, and 10 mg GDH powder were reacted at 28° C. with a substrate concentration of 10 mg/ml to 90 mg/ml. A TLC dot plate was used to determine the reaction progress. After 24 hours, a saturated sodium hydroxide solution was added to adjust the pH value to above 10, and the denatured protein was removed through centrifugation. The supernatant was extracted with dichloromethane, dried, and spin-dried to collect the product, and then HPLC detection was performed. See, Table 2.

TABLE 2

| | Substrate | Coenzyme | Conversion | ee |
|---|---|---|---|---|
| No. | concentration | NADP⁺ | rate | (%) |
| 1 | 10 | 0 | 45.4 | 99.7 |
| 2 | 10 | 0.2 | 99.8 | 99.8 |
| 3 | 20 | 0.2 | 99.8 | 99.7 |
| 4 | 30 | 0.2 | 79.7 | 99.8 |
| 5 | 30 | 0.4 | 99.7 | 99.8 |
| 6 | 40 | 0.4 | 99.5 | 99.7 |
| 7 | 50 | 0.4 | 89.6 | 99.7 |
| 8 | 50 | 0.6 | 99.4 | 99.7 |
| 9 | 60 | 0.6 | 98.5 | 99.7 |
| 10 | 70 | 0.8 | 96.3 | 99.7 |
| 11 | 80 | 0.8 | 94.9 | 99.7 |
| 12 | 90 | 0.9 | 60.4 | 99.7 |

Table 2 illustrates that the conversion rate was higher when the substrate concentration and the coenzyme NADP⁺ percentage were 10 to 20 and 0.2 respectively. Further, the conversion rate was significantly higher when the substrate concentration and the coenzyme NADP⁺ percentage were 30 to 60 and 0.4 to 0.6 respectively.

### Example 5: Synthesis of (S)-2-(3-Pyridyl)-Pyrrolidine Through Catalysis of the Crude Enzyme Solution of Imine Reductases

A 10 ml phosphate buffer solution (pH 7.5), a 60 mg/ml crude enzyme solution obtained by breaking imine reductase bacteria (i.e., subjecting the recombinant bacteria of Example 1 to fermenting, concentrating, resuspending with a buffer, and crushing by a high-pressure homogenizer to obtain the crude enzyme solution), 2 eq glucose, 0 mg/ml to 0.8 mg/ml NADP⁺, and 10 mg GDH powder were reacted at 30° C. with a substrate concentration of 10 mg/ml to 90 mg/ml. A TLC dot plate was used to determine the reaction progress. After 24 hours, a saturated sodium hydroxide solution was added to adjust the pH value to above 10, and the denatured protein was removed through centrifugation. The supernatant was extracted with dichloromethane, dried, and spin-dried to collect the product, and then HPLC detection was performed. See, Table 3.

TABLE 3

| | Substrate | Coenzyme | Conversion | ee |
|---|---|---|---|---|
| No. | concentration | NADP⁺ | rate | (%) |
| 1 | 10 | 0 | 21.6 | 99.7 |
| 2 | 10 | 0.2 | 99.2 | 99.8 |
| 3 | 20 | 0.2 | 97.8 | 99.7 |
| 4 | 30 | 0.3 | 94.5 | 99.8 |
| 5 | 30 | 0.4 | 99.3 | 99.8 |
| 6 | 40 | 0.4 | 99.2 | 99.7 |
| 7 | 50 | 0.4 | 83.4 | 99.7 |
| 8 | 50 | 0.6 | 89.4 | 99.7 |
| 9 | 60 | 0.6 | 88.5 | 99.7 |
| 10 | 70 | 0.8 | 76.3 | 99.7 |
| 11 | 80 | 0.8 | 64.9 | 99.7 |

### Example 6: Synthesis of (S)-2-(3-Pyridyl)-Pyrrolidine Through Imine Reductase Whole-Cell Catalysis

A 1 L phosphate buffer solution (pH 7.5), 40 mg/ml imine reductase recombinant bacteria of Example 1, 2 eq glucose, 0.4 mg/ml NADP⁺, and 100 mg GDH powder were reacted at 28° C. with substrates added in batches to make a final substrate concentration of 50 mg/ml. A TLC dot plate was used to determine the reaction progress. After the reaction was completed, a saturated sodium hydroxide solution was added to adjust the pH value to above 10, the denatured protein was removed through centrifugation, and the supernatant was extracted with dichloromethane, dried, and spin-dried to collect the product. The yield was 96%, the purity was 98%, and the e.e. value was 99.8%.

### Example 7: Synthesis of (S)-2-(3-Pyridyl)-Pyrrolidine Through Imine Reductase Whole-Cell Catalysis

A 1 L phosphate buffer solution (pH 7.5), 40 mg/ml imine reductase recombinant bacteria of Example 1, 2 eq glucose, 0.5 mg/ml NADP+, and 100 mg GDH powder were reacted at 30° C. with substrates added in batches to make a final substrate concentration of 70 mg/ml. The TLC dot plate was used to determine the reaction progress. After the reaction was completed, a saturated sodium hydroxide solution was added to adjust the pH value to above 10, the denatured protein was removed through centrifugation, and the supernatant was extracted with dichloromethane, dried, and spin-dried to collect the product. The yield was 93%, the purity was 98%, and the e.e. value was 99.6%.

### Example 8: Synthesis of (S)-2-(3-Pyridyl)-Pyrrolidine Through Catalysis of the Crude Enzyme Solution of Imine Reductases

A 1 L phosphate buffer solution (pH 7.5), a 60 mg/ml crude enzyme solution obtained by breaking imine reductase bacteria (i.e., subjecting the recombinant bacteria of Example 1 to fermenting, concentrating, resuspending with a buffer, and crushing by a high-pressure homogenizer to obtain the crude enzyme solution), 2 eq glucose, 0.5 mg/ml NADP⁺, and 100 mg GDH powder were reacted at 30° C. with substrates added in batches to make a final substrate concentration of 50 mg/ml. A TLC dot plate was used to determine the reaction progress. After the reaction was completed, a saturated sodium hydroxide solution was added to adjust the pH value to above 10, the denatured protein was removed through centrifugation, and the supernatant was extracted with dichloromethane, dried, and spin-dried to collect the product. The yield was 94%, the purity was 98%, and the e.e. value was 99.7%.

### Example 9: Large-Scale Fermentation Process and Whole-Cell Catalysis

### 1. Strain Preservation

A selected single colony was coated on a plate containing LB medium and cultured overnight at 37° C. Then, the colonies on the plate were scraped and inoculated in the shake flask with LB medium. The shake flask was cultured in an incubator at 37° C., 200 rpm for 12 h. The bacteria liquid was mixed with 40% glycerol in equal volumes and stored at minus 20° C., when OD600 was above 2.0. (LB medium is shown below in Table 4).

TABLE 4
LB medium content

| | | | | Ratio of | |
|---|---|---|---|---|---|
| Material | Specification | Manufacturer | Batch No. | Materials | |
| Tryptone | 500 g AR | OXOID | 02813 | 10.00 | g/L |
| Yeast Extract | 500 g AR | OXOID | 07852 | 5.00 | g/L |
| NaCl | 500 g AR | FuChen | 20200202 | 10.00 | g/L |
| Kanamycin | 5 g BR | Aladdin | J1909216 | 0.05 | g/L |
| AGAR* | 250 g BR | AOBOX | 20191223 | 10 | g/L |

pH Adjusted to 7.0 with ammonia water before sterilization
*In embodiments with liquid cultures, agar is omitted

### 2. Seed Culture

1 ml of bacterial liquid was taken from the glycerol stock and inoculated into shake flask with 400 ml LB medium, and then, the flask was cultured in an incubator. The formula of medium and culture condition are shown below in Tables 5 and 6.

TABLE 5
Formula of Shake flask Medium

| | | Manufac- | | Ratio of | |
|---|---|---|---|---|---|
| Material | Specification | turer | Batch No. | Materials | Mass |
| Tryptone | 500 g AR | OXOID | 02813 | 10.00 g/L | 4.10 g |
| Yeast | 500 g AR | OXOID | 07852 | 5.00 g/L | 2.05 g |
| Extract | | | | | |
| NaCl | 500 g AR | FuChen | 20200202 | 10.00 g/L | 4.06 g |
| Kanamycin | 5 g BR | Aladdin | J1909216 | 0.05 g/L | 0.02 g |

Volume: 400 mL;
pH Adjusted to 7.0 with ammonia water before sterilization

TABLE 6
Culture Conditions of Shake flask

| Medium volume | Sterilization conditions | Inoculation amount |
|---|---|---|
| 400 ml/2000 ml | 121° C., 30 min | 0.25% |
| Temperature | Rotation speed | Culture Cycle |
| 37° C. | 200 rpm | 10 h |

### 3. Fermentation

The seed liquid was inoculated in a bioreactor with 25 L culture medium, when OD600 was above 2.0. The formula of fermentation medium and conditions of sterilization and culture were shown below in Table 7. The fermentation liquid was cooled to 28° C. when OD600 was above 2.0. This operation occurred about 4 hours after inoculation, as seen in Fermentation Record shown in Table 8.

TABLE 7
Formula of Fermentation Medium

| | | | Ratio of | |
|---|---|---|---|---|
| | | | Materials | Mass |
| Material | Specification | Manufacturer | (g/L) | (g) |
| Peptone | FP102 | Angela. | 12 | 301.00 |
| Yeast Extract | FM902 | Angela. | 5 | 125.00 |
| Alpha lactose | 500 g AR | Sinopharm | 10 | 250.00 |
| NaCl | 500 g AR | FuChen | 5 | 126.00 |
| K₂HPO₄•3H₂O | 500 g AR | FuChen | 3 | 75.00 |
| KH₂PO₄ | 500 g AR | GuangFu | 1.5 | 38.00 |
| MgSO₄•7H₂O | 500 g AR | FuChen | 0.41 | 10.40 |
| Defoaming | TAI-X-298 | HengXin | 0.13 | 3.30 |
| agent | | | | |
| Kanamycin | 5 g BR | Aladdin | 0.05 | 1.25 |

Volume: 25 L;
pH Adjusted to 7.2-7.5 with ammonia water before sterilization

TABLE 8
Fermentation Record

| | | DO | | Temperature | | Air Flow | Pressure |
|---|---|---|---|---|---|---|---|
| Period | Agitation | (%) | OD600 | (° C.) | pH | (m³/h) | (Mpa) |
| 0 | 200 | 100.0 | 0.13 | 37 | 6.71 | 0.75 | 0.05 |
| 1 | 200 | 93.2 | | 37 | 6.69 | 0.75 | 0.05 |
| 2 | 200 | 83.5 | | 37 | 6.67 | 0.75 | 0.05 |
| 3 | 200 | 49.7 | 0.85 | 37 | 6.56 | 0.75 | 0.05 |
| 4* | 200 | 24.3 | | 28 | 6.55 | 0.75 | 0.05 |
| 5 | 200 | 38.6 | 3.4 | 28 | 6.56 | 0.75 | 0.05 |
| 6 | 220 | 3.0 | | 28.1 | 6.87 | 0.75 | 0.05 |
| 7 | 220 | 3.7 | | 28.1 | 7.11 | 0.75 | 0.05 |
| 8 | 240 | 5.3 | | 28 | 7.24 | 0.75 | 0.05 |
| 9 | 240 | 46.5 | 10.65 | 28.1 | 7.40 | 0.75 | 0.05 |
| 10 | 240 | 63.1 | | 28.1 | 7.49 | 0.75 | 0.05 |
| 11 | 240 | 65.5 | | 28 | 7.51 | 0.75 | 0.05 |
| 12 | 240 | 72.0 | 16 | 28.1 | 7.53 | 0.75 | 0.05 |
| 13 | 200 | 62.0 | | 28 | 7.51 | 0.75 | 0.05 |
| 14 | 200 | 64.0 | 13.6 | 28 | 7.54 | 0.75 | 0.05 |
| 15 | 180 | 63.5 | | 28.1 | 7.62 | 0.75 | 0.05 |
| 16 | 180 | 66.0 | | 28 | 7.64 | 0.75 | 0.05 |
| 17 | 180 | 72.3 | | 28 | 7.69 | 0.75 | 0.05 |
| 18 | 180 | 73.3 | | 28.1 | 7.74 | 0.75 | 0.05 |
| 19 | 180 | 74.1 | 12.4 | 28 | 7.77 | 0.75 | 0.05 |

Inoculation Volume: 280 ml;
OD600 of the seed liquid: 2.9;
pH of the seed liquid: 7.55;
*↓28° C.;
Volume Before Sterilization: 23 L;
pH Before Sterilization: 7.28;
Temperature of Sterilization: 115° C.;
Volume After Sterilization: 25 L;
pH After Sterilization: 6.69;
Time of Sterilization: 30 min;
Fermenter: F2

### Example 9: Obtaining High-Expression Genetically Engineered Bacteria

Whole gene synthesis was completed by General Biosystems (Anhui) Co., Ltd.

Codon optimization was performed on imine reductases MsIR1 (WP_074958336.1) of the bacterium (Myxococcus fulvus) (SEQ ID NO: 2), so that the gene could be expressed in an Escherichia coli expression host. Nde I and EcoR I restriction enzyme cutting sites were added at both ends of the gene to allow for insertion into the pET-28a(+) vector to obtain genetically engineered bacteria M1.

The prepared recombinant vector was transformed into Escherichia coli BL21, ROSETTA™, or ORIGAMI™ using conventional methods to yield genetically engineered bacteria in which the recombinant imine reductases were present in a soluble form, and successfully constructed genetically engineered bacteria were identified. The target protein was better expressed in the recombinant Escherichia coli BL21 host. Engineered bacteria with a target protein expression level of not less than 20% were used as an engineered bacterial strain for production and were stored in glycerol or freeze-dried milk.

### Example 10: Genetically Engineered Bacteria Culture and Crude Enzyme Solution Preparation

A single plated colony of the recombinant bacteria of Example 1 was inoculated into a 5 ml fermentation culture medium containing corresponding antibiotics for about 15 h of culture to obtain a seed solution. The seed solution was inoculated into a 600 ml fermentation culture medium with an inoculation amount of 1% and then cultured on a 200-rpm shaker at 37° C. to OD600=0.6 to 0.8. IPTG with a final concentration of 0.1 mM was added for induction for more than 10 h. The culture solution was centrifuged at 8000 rpm to collect bacteria, and high pressure breakage was performed to obtain a crude enzyme solution of the imine reductases.

### Example 11

After 300 g of methyl nicotinate, 300 g of vinylpyrrolidone, and 340 g of potassium tert-butoxide were added to dry n-hexane, reflux stirring was performed for 3 hours. After room temperature was reached through cooling, filtering was performed, the resulting filter cake was added to dilute hydrochloric acid, and reflux stirring was performed for 9 hours. After cooling, a base was added to adjust pH to 10, and extraction was performed by using ethyl acetate. After concentration, a catalytic reaction was carried out using the enzyme system of Example 2 for 14 hours. The pH was adjusted to 10 after a methylation reaction using formaldehyde and formic acid, extraction was performed using ethyl acetate, and 283.8 g of (S)-nicotine was obtained after concentration and distillation. A total yield of 80.6%, GC purity of 99.69%, and optical purity of 99% e.e. of (S)-nicotine was obtained.

### Example 12

After 150 g of ethyl nicotinate, 132.4 g of vinylpyrrolidone, and 35.8 g of sodium hydride were added to dry n-hexane, reflux stirring was performed for 5 hours. After room temperature was reached through cooling, filtering was performed, the resulting filter cake was added to dilute hydrochloric acid, and reflux stirring was performed for 16 hours. After cooling, a base was added to adjust the pH to 14, and extraction was performed using n-hexane. After concentration, a catalytic reaction was carried out using the enzyme system of Example 2 for 8 hours. The pH was then adjusted to 14 after a methylation reaction using formaldehyde and formic acid, extraction was performed by using n-hexane, and 136.7 g of (S)-nicotine was obtained after concentration and distillation. A total yield of 85%, GC purity is 99.7%, and optical purity of 99% e.e. of (S)-nicotine was obtained.

### Example 13

After 300 g of ethyl nicotinate, 280 g of vinylpyrrolidone, and 300 g of potassium tert-butoxide were added to dry n-hexane, reflux stirring was performed for 3 hours. After room temperature was reached through cooling, filtering was performed, the resulting filter cake was added to dilute hydrochloric acid, and reflux stirring was performed for 21 hours. After cooling, a base was added to adjust the pH to 12, and extraction was performed using dichloromethane. After concentration, a catalytic reaction was carried out using the enzyme system of Example 2 for 12 hours, the pH was adjusted to 12 after a methylation reaction using formaldehyde and formic acid, an extraction was performed using dichloromethane, and 261 g of (S)-nicotine was obtained after concentration and distillation. A total yield of 81.1%, GC purity of 99.7%, and optical purity of 99% e.e. of (S)-nicotine was obtained.

### Example 14

After 200 g of tert-butyl nicotinate, 175 g of vinylpyrrolidone, and 160.9 g of sodium tert-butoxide were added to dry n-hexane, reflux stirring was performed for 2 hours. After room temperature was reached through cooling, filtering was performed, the resulting filter cake was added to dilute hydrochloric acid, and reflux stirring was performed for 30 hours. After cooling, a base was added to adjust the pH to 9, and extraction was performed using chloroform. After concentration, a catalytic reaction was carried out using the enzyme system for 16 hours, the pH was adjusted to 9 after a methylation reaction using formaldehyde and formic acid, extraction was performed using chloroform, and 143 g of (S)-nicotine is obtained after concentration and distillation. A total yield of 79.1%, GC purity of 99.62%, and optical purity of 99% e. e of (S)-nicotine was obtained.

### Example 15

After 200 g of methyl nicotinate, 190.7 g of vinylpyrrolidone, and 120 g of lithium tert-butoxide were added to dry n-hexane, reflux stirring was performed for 5 hours. After room temperature was reached through cooling, filtering was performed, the resulting filter cake was added to dilute hydrochloric acid, and reflux stirring was performed for 30 hours. After cooling, a base was added to adjust the pH to 11, and extraction was performed using n-hexane. After concentration, a catalytic reaction was carried out using the enzyme system of Example 2 for 8 hours, the pH was adjusted to 11 after a methylation reaction using formaldehyde and formic acid, extraction was performed using n-hexane, and 190 g of (S)-nicotine was obtained after concentration, and distillation. A total yield of 80%, GC purity of 99.69%, and optical purity of 99% e.e. of (S)-nicotine was obtained.

### Example 16

After 300 g of tert-butyl nicotinate, 242 g of vinylpyrrolidone, and 288 g of potassium tert-butoxide are added to dry toluene, reflux stirring was performed for 5 hours, After room temperature was reached through cooling, filtering was performed, the resulting filter cake was added to dilute hydrochloric acid, and reflux stirring was performed for 30 hours. After cooling, a base was added to adjust the pH to 9, and extraction was performed using dichloromethane. After concentration, a catalytic reaction was carried out using the enzyme system of Example 2 for 16 hours, the pH was adjusted to 9 after a methylation reaction using formaldehyde and formic acid, extraction was performed using dichloromethane, and 215 g of (S)-nicotine was obtained after concentration, and distillation. A total yield of 79.1%, GC purity of 99.62%, and optical purity of 99% e. e of (S)-nicotine was obtained.

### Example 17: Process for Preparing Nicotine

(i) Condensation Step

A reaction flask was replaced by nitrogen, and then n-hexane (325 g), ethyl nicotinate (225 g) and N-vinyl-2-pyrrolidone (198 g) were added in sequence to prepare a mixed solution, and the mixed solution was heated to 25-30° C. for later use. Another reaction flask was replaced with nitrogen, and then n-hexane (1625 g) and t-BuOK (250 g) were added, and then the mixture was heated to 45-50° C. The mixed solution containing the nicotinate and pyrrolidone was added to the mixture containing the t-BuOK dropwise, and then the reaction solution was heated to 55-65° C. for 3 hours. The reaction solution was cooled to below 40° C., and Intermediate 1 was obtained by filtration.

(ii) Cyclization Step

Water (1300 g) and hydrochloric acid (1200 g) were added to a reaction flask, and then Intermediate 1 was added with stirring. The mixture was heated to 95-105° C. for 12 hours, and then the mixture was cooled to below 50° C. Sodium hydroxide solution was added to the reaction solution until the pH was adjusted to 10-12. Dichloromethane (2500 g) was added for extraction, and then the organic phase was concentrated in vacuo to obtain Intermediate 2

(iii) Reduction Step

Glucose (270 g) and NADP-2Na (1.2 g) were added to the enzyme solution (4200 g) of Example 2. After full stirring, the mixture was heated to 28-35° C. Intermediate 2 was dropwise added to the mixture for 11 hours, and the temperature retained for another hour. The reaction solution was used for the next reaction.

(iv) Methylation Step

Formaldehyde aqueous solution (180 g) and formic acid aqueous solution (95.5 g) were added to the reaction solution of Step (iii). After full stirring, the mixture was heated to 60-65° C. for 3 hours with stirring. The reaction solution was cooled to below 40° C., filtered, and then dichloromethane (2400 g) was added for extraction. The organic phase was concentrated in vacuo to obtain the crude nicotine.

(v) Distillation Step

The crude nicotine was added to a reaction flask, and the temperature was raised to 60° C. The crude nicotine was distilled under reduced pressure. The front cut fraction was collected until the temperature reached 110° C. When the temperature reached 110° C., the nicotine was collected under reduced pressure until the temperature reached 125° C.

The foregoing descriptions are merely preferred embodiments of the present disclosure and are further detailed descriptions made for the present disclosure with reference to the specific preferred implementations. It should be understood that the specific implementations of the present disclosure are not limited to these descriptions. Any modifications, equivalent replacements, or improvements made without departing from the spirit and principle of the disclosure shall fall within the protection scope of the present disclosure.

Sequence Listing

| | | SEQ |
|---|---|---|
| Description | Sequence | ID NO: |
| Codon | ATGAAGCCGCATATTAGTATTCTGGGTGCAGGTCGTATGGG | 1 |
| optimized | CAGTGCCCTGGTGAAAGCATTTCTGCAGAATGAATATACCA | |
| nucleotide | CCACCGTTTGGAATCGTACCCGTGCACGTTGTGAACCGCTG | |
| sequence | GCAGCAGCAGGCGCCCGTATTGCCGATAGCGTTCGCGATGC | |
| encoding | AGTGCAGACCGCCAGCGTGGTTATTGTGAATGTGAATGATT | |
| imine | ATGATACCAGCGATGCCCTGCTGCGCCAGGATGAAGTGACC | |
| reductase of | CAGGAACTGCGCGGTAAAGTTCTGGTGCAGCTGACCAGCGG | |
| Myxococcus | CAGTCCGAAACTGGCCCGCGAACAGGCCACCTGGGCCAGAC | |
| fulvus | GTCATGGTATTGATTATCTGGATGGTGCAATTATGGCCACCC | |
| | CGGATCTGATTGGTCGTCCGGATTGTACCCTGCTGTATGCCG | |
| | GCCCGAAAGCACTGTATGATAAACATCAGGCCGTTCTGGCA | |
| | GCACTGGGTGGCAATACCCAGCATGTTAGTGAAGATGAAGG | |
| | TCATGCAAGCGCACTGGATAGTGCCATTCTGTTTCAGCTGTG | |
| | GGGTAGCCTGTTTAGTGGTCTGCAGGCCGCCGCAATTTGTCG | |
| | TGCAGAAGGCATTGCCCTGGATGCACTGGGTCCGCATCTGG | |
| | AAGCAGTGGCCGCCATGATTCAGTTTAGCATGAAAGATCTG | |
| | CTGCAGCGTATTCAGAAAGAACAGTTTGGTGCAGATACCGA | |
| | AAGCCCGGCAACCCTGGATACCCATAATGTTGCCTTTCAGC | |
| | ATCTGCTGCATCTGTGCGAAGAACGTAATATTCATCGCGCCC | |
| | TGCCGGAAGCAATGGATGCACTGATTCAGACCGCACGCAAA | |
| | GCCGGTCATGGCCAGGATGATTTTAGTGTTCTGGCACGTTTT | |
| | CTGCGTTAA | |
| Wild type | MKPHISILGAGRMGSALVKAFLQNEYTTTVWNRTRARCEPLA | 2 |
| imine | AAGARIADSVRDAVQTASVVIVNVNDYDTSDALLRQDEVTQE | |
| reductase of | LRGKVLVQLTSGSPKLAREQATWARRHGIDYLDGAIMATPDLI | |
| Myxococcus | GRPDCTLLYAGPKALYDKHQAVLAALGGNTQHVSEDEGHAS | |
| fulvus | ALDSAILFQLWGSLFSGLQAAAICRAEGIALDALGPHLEAVAA | |
| | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSAAVKAFLQNEYTTTVWNRTRARCDPLA | 3 |
| reductase, | AAGARIADSVRDAVQTASVVIVNVNDDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSAAVKALQNEYTTTVWNRTRARCEPLA | 4 |
| reductase, | AAGARIADSVRDAVQTASVVIVNVFDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALTKAFAQNEYTTTVWNRTRARCEPLA | 5 |
| reductase, | AAGARIADSVRDAVQTASVVIVNVFDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALTKAFLQNEYTTTVWNRTRARCDPLA | 6 |
| reductase, | AAGARIADSVRDAVQTASVVIVNVNDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFAQNEYTTTVWNRTRARCEPLA | 7 |
| reductase, | AAGARIADSVRDAVQTASVVIENVNDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFAQNEYTTTVWNRTRARCEPLA | 8 |
| reductase, | AAGARIADSVRDAVQTASVVIENVNDYDTSDALLRQDEVTQE | |
| mutated | LRGKVLVQTTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCAPLA | 9 |
| reductase, | AAGARIADSVRDAVQTASVVIVNVNDYDTSDALLRQDEVTQE | |
| mutated | LRGKVLVQLTSGSPKLAREQATWARRHGIWYLDGAIMATPDL | |
| residues bold | IGRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCGPLA | 10 |
| reductase, | AAGARIADSVRDAVQTASVVIVNVNDYDTSDALLRQDEVTQE | |
| mutated | LRGKVLVQTTSGSPKLAREQATWARRHGPWYLDGAIMATPDL | |
| residues bold | IGRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 11 |
| reductase, | AGGARIADSVRDAVQTASVVIVNVNDYDTSDALLRQDEVTQE | |
| mutated | LRGKVLVQTTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 12 |
| reductase, | AGGARIADSVRDAVQTASVVIENVFDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 13 |
| reductase, | AAGARIADSVRDAVQTASVVIENVFDYDTSDALLRQDEVTQE | |
| mutated | LRGKVLVQLTSGSPKLAREQATWARRHGIDYLDGAIMAWPDL | |
| residues bold | IGRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHNVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 14 |
| reductase, | AAGARIADSVRDAVQTASVVIVNVFDYDTSDALLRQDEVTQE | |
| mutated | LRGKVLVQKTSGSPKLAREQATWARRHGIDYLDGAIMAWPD | |
| residues bold | LIGRPDCTLLYAGPKALYDKHQAVLAALGGNTQHVSEDEGHA | |
| and | SALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 15 |
| reductase | AAGARIADSVRDAVQTASVVIENVFDYDTSDALLRQDEVTQE | |
| mutated | LRGKVLVQLTSGSPKLAREQATWARRHGIDYLDGAIMAFPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 16 |
| reductase, | AAGARIADSVRDAVQTASVVIVNVFDYDTSDALLRQDEVTQE | |
| mutated | LRGKVLVQLTSGSPKLAREQATWARRHGIWYLDGAIMAFPDL | |
| residues bold | IGRPDCTLLYAGPKALYDKHQAVLAALGGNTQHVSEDEGHAS | |
| and | ALDSAILFQLWGSLFSGLQAAAICRAEGIALDALGPHLEAVAA | |
| underlined | MIQFSMKDLLQRIQKEQFGADTESPATLDTHLVAFQHLLHLCE | |
| | ERNIHRALPEAMDALIQTARKAGHGQDDFSVLARFLR | |
| Mutant imine | MKPHISILGAGRMGSALVKAFLQNEYTTTVWNRTRARCEPLA | 17 |
| reductase, | AAGARIADSVRDAVQTASVVIVNVNDYDTSDALLRQDEVTQE | |
| mutated | LRGKVLVQKTSGSPKLAREQATWARRHGIDYLDGAIMAFPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAAS | |
| underlined | IQFSMKDLLQRIQKEQFGADTESPATLDTHLVAFQHLLHLCEE | |
| | RNIHRALPEAMDALIQTARKAGHGQDDFSVLARFLR | |
| Mutant imine | MKPHISILGAGRMGSALVKAFLQNEYTTTVWNRTRARCEPLA | 18 |
| reductase, | AAGARIADSVRDAVQTASVVIVNVNDYDTSDALLRQDEVTQE | |
| mutated | LRGKVLVQKTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAAS | |
| underlined | IQFSMKDLLQRIQKEQFGADTESPATLDTHLVAFQHLLHLCEE | |
| | RNIHRALPEAMDALIQTARKAGHGQDDFSVLARFLR | |
| Mutant imine | MKPHISILGAGRMGSALVKAFLQNEYTTTVWNRTRARCEPLA | 19 |
| reductase, | AAGARIADSVRDAVQTASVVIVNVNDYDTSDALLRQDEVTQE | |
| mutated | LRGKVLVQLTSGSPKLAREQATWARRHGIDYLDGAIMATPDLI | |
| residues bold | GRPDCTLLYAGPKALYDKHQAVLAALGGNTQHVSEDEGHAS | |
| and | ALDSAILFQLWGSLFSGLQAAAICRAEGIALDALGPHLEAVAAS | |
| underlined | IQFSMKDLLQRIQKEQFGADTESPATLDTHLVAFQHLLHLCEE | |
| | RNIHRALPEAMDALIQTARKAGHGQDDFSVLARFLR | |
| NZ_ | TCAGCGCAGGAAGCGCGCGAGGACCGAGAAGTCGTCCTGG | 20 |
| FOIB01000013 | CCATGCCCCGCCTTCCGGGCGGTCTGGATGAGTGCATCCAT | |
| Region: | GGCCTCGGGGAGAGCGCGGTGGATGTTGCGCTCCTCGCACA | |
| 55611..56486, | GGTGCAGCAGGTGCTGGAACGCCACGTTGTGCGTGTCGAGC | |
| negative | GTGGCGGGGCTCTCGGTGTCCGCGCCGAACTGCTCCTTCTGG | |
| strand | ATGCGCTGGAGGAGGTCCTTCATGCTGAACTGAATCATGGC | |
| | CGCGACGGCCTCCAGATGCGGGCCGAGCGCGTCGAGCGCAA | |
| | TCCCCTCGGCGCGGCAGATGGCCGCGGCCTGCAGCCCGCTG | |
| | AACAGCGAACCCCACAGCTGGAACAGGATGGCGCTGTCGA | |
| | GCGCGGACGCGTGGCCCTCGTCCTCGCTCACGTGCTGCGTGT | |
| | TTCCACCGAGCGCCGCCAGCACGGCCTGGTGCTTGTCGTAC | |
| | AGGGCCTTCGGGCCCGCGTACAGGAGCGTGCAGTCGGGCCG | |
| | GCCGATGAGGTCCGGCGTGGCCATGATGGCGCCGTCCAGGT | |
| | AGTCGATGCCGTGCCGTCGTGCCCACGTCGCCTGCTCGCGC | |
| | GCCAGCTTCGGTGAGCCGGACGTGAGCTGCACCAGCACCTT | |
| | GCCCCGGAGCTCCTGCGTCACCTCGTCCTGGCGCAGCAGCG | |
| | CGTCGCTGGTGTCGTAGTCATTCACGTTCACGATGACGACGC | |
| | TGGCTGTCTGCACCGCGTCTCGCACGGAGTCGGCGATGCGC | |
| | GCGCCCGCTGCCGCCAACGGCTCGCACCGCGCCCGGGTGCG | |
| | ATTCCAGACCGTGGTCGTGTACTCGTTCTGGAGGAACGCCTT | |
| | GACCAGCGCGGAGCCCATGCGGCCCGCGCCGAGGATGCTGA | |
| | TGTGTGGCTTCAT | |
| reverse | ATGAAGCCACACATCAGCATCCTCGGCGCGGGCCGCA | 21 |
| complement | TGGGCTCCGCGCTGGTCAAGGCGTTCCTCCAGAACGAG | |
| nucleotide | TACACGACCACGGTCTGGAATCGCACCCGGGCGCGGT | |
| sequence of | GCGAGCCGTTGGCGGCAGCGGGCGCGCGCATCGCCGA | |
| SEQ ID NO: | CTCCGTGCGAGACGCGGTGCAGACAGCCAGCGTCGTC | |
| 20 | ATCGTGAACGTGAATGACTACGACACCAGCGACGCGC | |
| | TGCTGCGCCAGGACGAGGTGACGCAGGAGCTCCGGGG | |
| | CAAGGTGCTGGTGCAGCTCACGTCCGGCTCACCGAAGC | |
| | TGGCGCGCGAGCAGGCGACGTGGGCACGACGGCACGG | |
| | CATCGACTACCTGGACGGCGCCATCATGGCCACGCCGG | |
| | ACCTCATCGGCCGGCCCGACTGCACGCTCCTGTACGCG | |
| | GGCCCGAAGGCCCTGTACGACAAGCACCAGGCCGTGC | |
| | TGGCGGCGCTCGGTGGAAACACGCAGCACGTGAGCGA | |
| | GGACGAGGGCCACGCGTCCGCGCTCGACAGCGCCATC | |
| | CTGTTCCAGCTGTGGGGTTCGCTGTTCAGCGGGCTGCA | |
| | GGCCGCGGCCATCTGCCGCGCCGAGGGGATTGCGCTC | |
| | GACGCGCTCGGCCCGCATCTGGAGGCCGTCGCGGCCAT | |
| | GATTCAGTTCAGCATGAAGGACCTCCTCCAGCGCATCC | |
| | AGAAGGAGCAGTTCGGCGCGGACACCGAGAGCCCCGC | |
| | CACGCTCGACACGCACAACGTGGCGTTCCAGCACCTGC | |
| | TGCACCTGTGCGAGGAGCGCAACATCCACCGCGCTCTC | |
| | CCCGAGGCCATGGATGCACTCATCCAGACCGCCCGGA | |
| | AGGCGGGGCATGGCCAGGACGACTTCTCGGTCCTCGC | |
| | GCGCTTCCTGCGCTGA | |
| codon | ATGGCCAGTATTTGTGTGGTGGGTGCAGGTCGTATGGG | 22 |
| optimized | TAGCGCCCTGGCACGTGCATTTCTGCGCGCAGGTTATG | |
| nucleotide | TGACCCATGTTTGGAATCGTACCCCGGCAAAAGGTGAA | |
| sequence | GCCCTGGCAGCACTGGGTGCCCGTTTTGTTCCGAGTCT | |
| encoding the | GCATCAGGCAATTGCCGCCAGCGATATTGTTGTTGTGA | |
| imine | ATGTGATTGATTACGCCGCCGCCGATGCACATCTGCGC | |
| reductase of | AGTGCCAGCGTGACCCGCGCCTTAGGTCGTAAACTGCT | |
| Bosea sp. | GGTGCAGCTGACCAGTGGCAGCCCGAGTCAGGCCCGC | |
| | CAGACCGGTGAATGGGCCAAAGGTCATGGCGTGGGTT | |
| | ATCTGGATGGCGCCATTATGGCCACCCCGAATTTTATT | |
| | GGCGAACCGAGTGCAACCATTCTGTATAGCGGTAGTCA | |
| | GCATGCCTTTGATGAAAATCGCGATGTGTTTCTGGCAC | |
| | TGGGCGGTAATGCCGTTCATGTTGGTGACGATTTTGGC | |
| | CATGCCAGCGCCCTGGATATTGCCCTGCTGAGCCAGCT | |
| | GTGGGGTACCCTGTTTGGTACCCTGCAGGCAATTGCGG | |
| | TGAGCCAGGCCGAAGGTATTGAACTGGATGCCTATGCC | |
| | CGCTATCTGCAGCCGTTTAAACCGACCATTGATGGTGC | |
| | CGTTGCCGATCTGGTTACCCGTGCCCGTGATGGTCGTT | |
| | ATCGCGGCGATGATCAGACCCTGGCAGCAATTGCCGC | |
| | ACATTATAGTGCCTTTCAGCCGCTGCTGGAAGTTAGCC | |
| | GCGAACGTGGCCTGAATCGCGCAGTGCCGGATGCATTT | |
| | GATAGTATTTTTAAAGCCGCCATTGCCGCAGGCCATCT | |
| | GCAGGATGATTTTGCCGCCCTGACCCGTTTTATGCGCT | |
| | AA | |
| imine | MASICVVGAGRMGSALARAFLRAGYVTHVWNRTPAKG | 23 |
| reductase of | EALAALGARFVPSLHQAIAASDIVVVNVIDYAAADAHLR | |
| Bosea sp. | SASVTRALGRKLLVQLTSGSPSQARQTGEWAKGHGVGY | |
| | LDGAIMATPNFIGEPSATILYSGSQHAFDENRDVFLALGG | |
| | NAVHVGDDFGHASALDIALLSQLWGTLFGTLQAIAVSQA | |
| | EGIELDAYARYLQPFKPTIDGAVADLVTRARDGRYRGDD | |
| | QTLAAIAAHYSAFQPLLEVSRERGLNRAVPDAFDSIFKAA | |
| | IAAGHLQDDFAALTRFMR | |

What is claimed is:

1. A nucleic acid molecule encoding an imine reductase of Myxococcus fulvus according to SEQ ID NO: 2 and comprising a nucleotide sequence codon optimized for heterologous expression.

2. The nucleic acid molecule of claim 1, wherein the heterologous expression is expression in a bacterial host cell.

3. The nucleic acid molecule of claim 1, wherein the heterologous expression is expression in Escherichia coli host cell selected from the group consisting of expression strains BL21, ROSETTA™, ORIGAMI™, and TUNER™ strains.

4. The nucleic acid molecule of claim 1 comprising a nucleotide sequence according to SEQ ID NO: 1.

5. The nucleic acid molecule of claim 1 in an expression vector.

6. The nucleic acid molecule of claim 5, wherein the expression vector comprises a nucleic acid molecule encoding glucose dehydrogenase.

7. A nucleic acid molecule encoding a mutant imine reductase of Myxococcus fulvus comprising an amino acid sequence according to any one of SEQ ID NOs: 3-19.

8. The nucleic acid molecule of claim 7 comprising a nucleotide sequence according to SEQ ID NOs: 1 and mutated to encode an amino acid sequence according to any one of SEQ ID NOs: 3-19.

9. The nucleic acid molecule of claim 7 in an expression vector.

10. The nucleic acid molecule of claim 9, wherein the expression vector comprises a nucleic acid molecule encoding glucose dehydrogenase.

11. A bacterial host cell comprising the nucleic acid molecule of claim 1.

12. A bacterial host cell comprising the nucleic acid molecule of claim 1 and an imine reductase having an amino acid sequence of any one of SEQ ID NOs: 2-19.

13. A crude enzyme solution comprising an imine reductase having an amino acid sequence of any one of SEQ ID NOs: 2-19.

14. A method of producing an imine reductase of Myxococcus fulvus according to SEQ ID NO: 2, or an imine reductase having an amino acid sequence of any one of SEQ ID NOs: 3-19, in bacterial host cells, the method comprising
a) culturing bacterial host cells comprising the nucleic acid molecule of claim 1 in a fermentation culture medium at a first temperature;
b) culturing the bacterial host cells in the fermentation culture medium at a second temperature; and
c) collecting the bacterial host cells.

15. A method of preparing 2-(3-pyridyl)-pyrrolidine, comprising combining the bacterial host cell of claim 11 with 2-pyridyl-1-pyrroline.

16. The method of claim 15, wherein the 2-(3-pyridyl)-pyrrolidine is (S)-2-(3-pyridyl)-pyrrolidine.

17. The method of claim 15, comprising combining the bacterial host cell of claim 11 with a buffer solution, and adding 2-pyridyl-1-pyrroline to the buffer solution.

18. The method of claim 17, wherein the buffer solution comprises a buffer selected from the group consisting of a phosphate buffer, Tris hydrochloride, bicarbonate buffer, and carbonate added to water.

19. The method of claim 15, wherein the reaction system comprises nicotinamide adenine dinucleotide phosphate (NADP+), glucose, and glucose dehydrogenase.

20. A method for preparing nornicotine, comprising contacting myosmine with the bacterial host cell of claim 11.

21. The method of claim 20, wherein the nornicotine is (S)-nornicotine.

22. The method of claim 20, further comprising methylating the nornicotine to provide nicotine.

23. The method of claim 22, wherein the methylating is performed using formaldehyde and formic acid.

24. The method of claim 22, wherein the nicotine is (S)-nicotine.

25. The method of claim 24, wherein the (S)-nicotine has an optical purity of at least about 95% ee, preferably at least about 99% ee.

26. The method of claim 20, further comprising contacting a 3-nicotinoyl-1-vinyl-4,5-dihydro-1H-pyrrol-2-olate salt with a dilute acid to provide the myosmine.

27. The method of claim 26, wherein the dilute acid is perchloric acid, sulfuric acid, hydrochloric acid, hydrobromic acid, phosphoric acid, trifluoromethanesulfonic acid, chlorosulfonic acid, sulfamic acid, trifluoroacetic acid, trichloroacetic acid, benzenesulfonic acid, and picric acid, or mixtures thereof, preferably hydrochloric acid.

28. The method of claim 26, wherein the 3-nicotinoyl-1-vinyl-4,5-dihydro-1H-pyrrol-2-olate salt is potassium 3-nicotinoyl-1-vinyl-4,5-dihydro-1H-pyrrol-2-olate or lithium 3-nicotinoyl-1-vinyl-4,5-dihydro-1H-pyrrol-2-olate.

29. The method of claim 26, wherein the pH of the myosmine is adjusted to about 10 to about 12.

30. The method of claim 26, further comprising combining a nicotinic acid ester and N-vinyl-2-pyrrolidone, and an alkali metal base to provide the 3-nicotinoyl-1-vinyl-4,5-dihydro-1H-pyrrol-2-olate salt.

31. The method of claim 30, wherein the nicotinic acid ester is methyl nicotinate, ethyl nicotinate, or tert-butyl nicotinate, preferably methyl nicotinate.

32. A method for preparing nicotine, comprising:
(i) combining a nicotinic acid ester and N-vinyl-2-pyrrolidone, and an alkali metal base to provide 3-nicotinoyl-1-vinyl-4,5-dihydro-1H-pyrrol-2-olate salt;
(ii) contacting the 3-nicotinoyl-1-vinyl-4,5-dihydro-1H-pyrrol-2-olate salt with a dilute acid to provide myosmine;
(iii) contacting the myosmine with the bacterial host cell of claim 11 to provide nornicotine; and
(iv) methylating the nornicotine to provide nicotine.

33. The method of claim 32, wherein the nornicotine is (S)-nornicotine.

34. Nicotine prepared according to the method of claim 32.

35. (S)-Nicotine prepared according to the method of claim 32.
